(19) United States
(12) Patent Application Publication
(10) Pub. No.: US 2007/0258986 A1
(43) Pub. Date: Nov. 8, 2007

(54) Targeted Delivery System for Bioactive Agents

(75) Inventors: Pradman Qasba, Bethesda, MD (US); Boopathy Ramakrishnan, Frederick, MD (US)

Correspondence Address: EDWARDS ANGELL PALMER & DODGE LLP, P.O. BOX 55874, BOSTON, MA, 02205, US

(73) Assignees: Govt of the US as represented by the secretary,; Govt of the US, as respresented by the secretary, Department of Health and Human Services, Rockville, MD (US)

(21) Appl. No.: 10/580,108

(22) PCT Filed: Nov. 18, 2004
(86) PCT No.: PCT/US04/38781

§ 371 Date: Feb. 13, 2007

Related U.S. Application Data
(60) Provisional application No. 60/523,112, filed on Nov. 19, 2003.

(51) Int. Cl. A61K 31/70; A61K 38/16; A61K 39/44; A61K 47/48; A61P 43/00; C12P 19/00

(52) U.S. Cl. 424/179.1; 435/72; 514/23; 514/62; 514/8; 530/387.1; 530/395; 536/1.11; 536/55.2

(57) ABSTRACT

In accordance with the present invention, compounds, compositions and methods are provided that allow for the administration of a bioactive agent to an organism, including a human or an animal. The present invention can be used to treat or prevent a disease and/or disorder with a bioactive agent, or can be used to safely vaccinate a human or animal against a bioactive agent. The invention can also be used as a method for the delivery of bioactive agents for the treatment or prevention of a disease and/or a disorder, particularly targeted delivery of bioactive agents through the administration of glycoconjugates containing a bioactive agent bound to a targeting compound through a modified saccharide residue.

### REFERENCE TO RELATED APPLICATIONS

This application claims priority under 35 U.S.C. 119(e) from U.S. Provisional Application Ser. No. 60/523,112, filed Nov. 19, 2003, which is incorporated herein by reference.

### GOVERNMENT FUNDING

The invention described herein was developed with support from the National Institutes of Health under contract N01-CO-12400. The U.S. Government may have certain rights in the invention.

### FIELD OF THE INVENTION

The present invention relates to novel targeted delivery systems for bioactive agents, and the use thereof. More particularly, the present invention relates to novel targeted delivery systems for bioactive agents including glycoconjugates including a bioactive agent joined to a targeting compound via a modified saccharide residue.

### BACKGROUND OF THE INVENTION

Current therapies for the treatment of diseases, disorders and pathological conditions, including genetic diseases, congenital diseases and other diseases including bacterial infections, viral infections, cancer, immune deficiency diseases, autoimmune diseases, psychiatric diseases, cardiovascular diseases, reproductive dysfunction, somatic growth dysfunction, stress related diseases, muscular dystrophy, osteoporosis, ocular diseases, allergies, and transplantation rejection, require administration of bioactive agents that have widespread effects throughout the body. Often, these effects reduce the quality of life of the patient and can be life threatening.

For example, current treatments for cancer include administration of chemotherapeutic agents, such as doxorubicin, and other bioactive agents such as cytokines and immune factors. The administration of chemotherapeutic agents to the entire body creates toxic and adverse side effects such as organ damage, including cardiotoxicity, loss of senses such as taste and feel, and hair loss. Many chemotherapeutic agents are designed to kill rapidly dividing cells which indiscriminately affects the hematopoetic system and the gastrointestinal system leading to changes in blood and immune cells, vomiting, gastric distress and weight loss. Administration of immune factors, such a cytokines, to the entire body leads to activation of unwanted immune responses and inhibition of other immune functions. Thus, such therapies provide treatment for the condition, but come with a wide array of side effects that must then be treated.

Most bioactive agents used in clinical settings are specific at a molecular rather than a cellular level. Moreover, generally only a small fraction of the dose reaches the target; the remaining amount of the bioactive agent acts on other tissues or is rapidly eliminated. This is usually a result of these agents not being specifically targeted/delivered to the affected cells, tissues or organs. Therefore, there is a need in the art for improved delivery systems for bioactive agents that may be used in therapies for a wide range of diseases and disorders, including immune diseases, cancers, cardiovascular diseases, viral diseases and bacterial diseases. More specifically, there is a need in the art for a bioactive agent delivery system capable of preferentially targeting therapeutically-relevant cells or tissues.

### SUMMARY OF THE INVENTION

The present invention relates to glycoconjugates in which a bioactive agent is bound through a modified saccharide residue, e.g., a modified galactose, to a compound which has an affinity for a target cell, for example, an antibody or antibody fragment which is specific to, for example, a cancer cell.

The present invention also provides a method for treatment of diseases and/or disorders by administration of one or more glycoconjugates of the invention. In particular, the present invention provides a method for the treatment of diseases and/or disorders through the targeted delivery of bioactive agents. The method includes administration of a composition containing a glycoconjugate having a bioactive agent linked to a targeting compound by a modified saccharide residue, e.g., a modified galactose residue having a ketone group. An advantage of this delivery system is that the bioactive agents are targeted to therapeutically-relevant cells and/or tissues. As such, a smaller amount of bioactive agent can be used than that with previously known methods. This yields reduced toxicity and fewer side-effects.

The invention can also be used to target a diagnostic agent such as a radioisotope, magnetic resonance imaging agent or ultrasound contrast agent to desired sites in the body. This would permit the diagnosis of diseases and/or disorders, including cancer, and also allow the extent of dissemination of the disease/disorder, such as cancer, through the body to be determined.

One embodiment provides a diagnostic compound for detecting a glycoprotein including a labeled modified sugar residue. A method of detecting a glycoprotein by introducing into a subject or a sample a detectable quantity of the diagnostic compound, allowing sufficient time for the labeled compound to become associated with the glycoprotein, and detecting the labeled compound associated with one or more glycoproteins is also provided.

One embodiment of the invention provides a targeted glycoconjugate comprising a bioactive agent and a targeting compound, wherein the bioactive agent and targeting compound are joined by a modified saccharide compound. Another embodiment of the invention provides pharmaceutical compositions comprising such glycoconjugates. Yet another embodiment of the invention provides kits comprising the glycoconjugates and/or pharmaceutical compositions comprising the glycoconjugates.

Another embodiment of the invention provides a method for the treatment or detection of a disease or disorder comprising, administering to a subject in need thereof a targeted glycoconjugate comprising a bioactive agent and a targeting compound, wherein the bioactive agent and targeting compound are joined by a modified saccharide compound.

Yet another embodiment of the invention provides a method of delivering one or more bioactive agents comprising administering to a subject a targeted glycoconjugate comprising a bioactive agent and a targeting compound, wherein the bioactive agent and targeting compound are joined by a modified saccharide compound.

One embodiment of the invention provides a method of vaccinating a human or animal against a bioactive agent. For example, a method of vaccinating a subject against a disease comprising administering to the subject an immunologically effective amount of a targeted glycoconjugate (a composition capable of generating an immune response) comprising a bioactive agent and a targeting compound, wherein the bioactive agent and targeting compound are joined by a modified saccharide compound is herein provided.

Another embodiment provides methods to synthesize the glycoconjugates of the invention. Also, one embodiment of the present invention provides for the use of the glycoconjugates in medical therapy and for the preparation of a medicament for the treatment of a disease or disorder.

### BRIEF DESCRIPTION OF THE FIGURES

FIG. 1 depicts an application of Y289L-Gal-T1 mutant for the efficient tagging of free GlcNAc moieties of glycoproteins, such as monoclonal antibodies (Ab). “X” represents any compound (e.g., a bioactive agent).

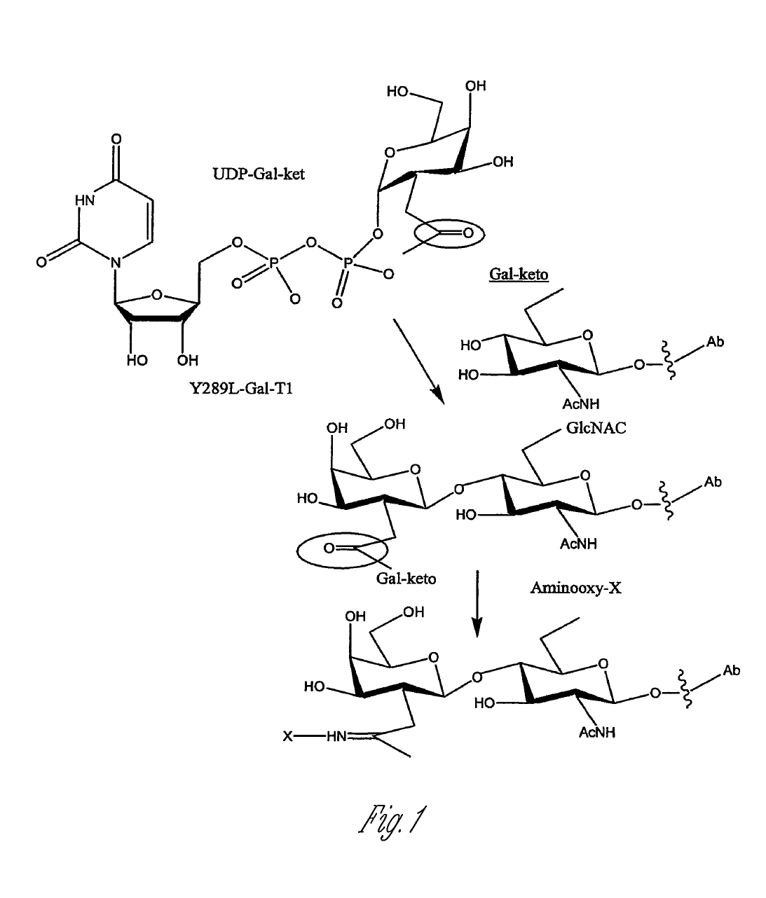

### DETAILED DESCRIPTION OF THE INVENTION

Targeted glycoconjugates and methods for their production and use are provided. Targeted glycoconjugates of the invention include a bioactive agent bound through a modified saccharide residue, e.g., a modified galactose, including a modified UDP-α-galactose, to a compound which has an affinity for a target cell, for example, an antibody or antibody fragment which is specific to, for example, a cancer cell.

A. Definitions

It is noted that, as used herein the singular forms “a,” “an,” and “the” include plural referents unless the context clearly dictates otherwise. Thus, for example, reference to a method for delivery of “a bioactive agent” or “a glycoconjugate” includes reference to delivery of a mixture of two or more bioactive agents or glycoconjugates. Thus, as used herein, the singular form may be used interchangeably with the plural form, and vice versa, i.e. “bioactive agent” could mean bioactive agents or “bioactive agents” could mean bioactive agent.

As used herein, “including” or “includes” or the like means including, without limitation.

As used herein, “organism” or “individual” or “subject” or “body” or “patient” refers to any animal, including mammals, preferably humans, or plant to which the present invention may be applied.

As used herein, “treat” or “treating” includes treating, preventing, ameliorating, or inhibiting a disease, disorder and/or a symptom of a disease and/or a disorder of an organism.

As used herein, “bioactive agent” means any chemical or biological material or compound suitable for delivery that induces a desired effect in or on an organism, such as a biological or pharmacological effect, which may include, but is not limited to, (1) having a prophylactic effect on the organism and preventing an undesired biological effect such as preventing an infection, (2) alleviating a condition caused by a disease or disorder, for example, alleviating pain or inflammation caused as a result of the disease or disorder, and/or (3) either alleviating, reducing, or completely eliminating the disease or disorder from the organism. As used herein, “bioactive agent” also refers to a substance which may be used in connection with an application that is therapeutic or diagnostic in nature, such as in methods for diagnosing the presence or absence of a disease or disorder in a patient and/or in methods for the treatment or prevention of a disease or disorder in a patient. As used herein, “bioactive agent” refers also to substances which are capable of exerting a biological effect in vitro and/or in vivo. Examples of suitable bioactive agents include diagnostic agents, pharmaceuticals, drugs, synthetic organic molecules, proteins, peptides, vitamins, steroids and genetic material, including nucleosides, nucleotides and polynucleotides.

As used herein, “genetic material” refers generally to nucleotides and polynucleotides, including deoxyribonucleic acid (DNA) and ribonucleic acid (RNA). The genetic material may be made by synthetic chemical methodology known to one of ordinary skill in the art, or by the use of recombinant technology, or by a combination of the two. The DNA and RNA may optionally comprise unnatural nucleotides and may be single, double or triple stranded. “Genetic material” refers also to sense and anti-sense DNA and RNA, that is, a nucleotide sequence which is complementary to a specific sequence of nucleotides in DNA and/or RNA.

As used herein, “saccharide” refers to any of a series of compounds of carbon, hydrogen, and oxygen in which the atoms of the latter two elements are in the ratio of 2:1, especially those containing the group C₆H₁₀O₅, including fructose, glucose, sucrose, lactose, maltose, galactose and arabinose. “Modified saccharide” refers to the modification of a saccharide by the attachment of a reactive functional group, including but not limited to, a ketone moiety.

As used herein, “pharmaceutical” or “drug” refers to any therapeutic or prophylactic bioactive agent which may be used in the treatment (including the prevention, diagnosis, alleviation, or cure) of a malady, affliction, disease, disorder or injury in a patient. Therapeutically useful peptides, polypeptides and polynucleofides may be included within the meaning of the term pharmaceutical or drug.

As used herein, an “effective amount” generally means a sufficient amount of a compound to provide the desired local or systemic effect and performance.

As used herein, “pharmaceutically acceptable carrier” refers to carrier materials without significant pharmacological activity at the quantities used that are suitable for administration with other compounds, and include any such materials known in the art, e.g., any liquid, gel, solvent, liquid diluents, solubilizer, microspheres, liposomes, microparticles, lipid complexes, or the like, that is sufficiently nontoxic at the quantities employed and does not interact with the drug to be administered in a deleterious manner. Examples of suitable carriers for use herein include water, buffers, mineral oil, silicone, inorganic or organic gels, aqueous emulsions, liquid sugars, lipids, microparticles, waxes, petroleum jelly, and a variety of other oils and polymeric materials.

As used herein, “covalent association” or “covalent bond” refers to an intermolecular association or bond which involves the sharing of electrons in the bonding orbitals of two atoms.

The term “acceptor” refers to a molecule or structure onto which a donor is actively linked through action of a catalytic domain of, for example, a galactosyltransferase, or mutant thereof. Examples of acceptors include, but are not limited to, carbohydrates, glycoproteins, and glycolipids.

The term “donor” refers to a molecule that is actively linked to an acceptor molecule through the action of a catalytic domain of, for example, a galactosyltransferase, or mutant thereof. A donor molecule can include a sugar, or a sugar derivative. Examples of donors include, but are not limited to, UDP-galactose, UDP-mannose, UDP-N-acetylglucosamine, UDP-glucose, GDP-mannose, UDP-N-acetylgalactosamine, UDP-glucuronic acid, GDP-Fucose, CMP-N-acetylneuraminic acid and/or modifications thereof. Donors include sugar derivatives that include active groups. Accordingly, oligosaccharides may be prepared according to the methods of the invention that include a sugar derivative having a desired characteristic.

As used herein, “targeting compound” refers to any material or substance which may promote targeting of tissues, cells and/or receptors in vivo and/or in vitro of the compounds/compositions of the present invention. The targeting compound may be synthetic, semi-synthetic, or naturally-occurring. Materials or substances which may serve as a targeting compound include, for example, ligands, proteins, including antibodies, glycoproteins and lectins, peptides, polypeptides, saccharides, including mono- and polysaccharides, vitamins, steroids, steroid analogs, hormones, cofactors, bioactive agents, and genetic material, including nucleosides, nucleotides and polynucleotides.

As used herein, “tissue” refers generally to specialized cells which may perform a particular function. It should be understood that the term “tissue,” as used herein, may refer to an individual cell or a plurality or aggregate of cells, for example, membranes or organs. The term “tissue” also includes reference to an abnormal cell or a plurality of abnormal cells. Exemplary tissues include, for example, myocardial tissue (also referred to as heart tissue or myocardium), including myocardial cells and cardiomyocites, plaques and atheroma, membranous tissues, including endothelium and epithelium, laminae, connective tissue, including interstitial tissue, lung, skin, pancreas, intestine, uterus, adrenal gland and retinal tissues, as well as tumors.

As used herein, “receptor” refers to a molecular structure within a cell or on the surface of the cell which is generally characterized by the selective binding of a specific substance (e.g., a ligand). Exemplary receptors include, for example, cell-surface receptors for peptide hormones, neurotransmitters, antigens, complement fragments, and immunoglobulins and cytoplasmic receptors for steroid hormones.

As used herein, “tumor cells” or “tumor” refers to an aggregate of abnormal cells and/or tissue which may be associated with diseased states that are characterized by uncontrolled cell proliferation. The disease states may involve a variety of cell types, including, for example, endothelial, epithelial and myocardial cells. Included among the disease states are neoplasms, cancer, leukemia and restenosis injuries.

The terms “toxic reaction” and “toxicity” as used herein, include, but are not limited to, the following responses of an animal or human: fever, edema, including cerebral edema, psychosis, autoimmune diseases, hemorrhage, shock, including hemorrhagic shock, sepsis, cachexia, or death.

B. Targeted Glecoconjugate Compounds

The glycoconjugate compounds of the invention are non-naturally occurring compounds that are a conjugate of a bioactive agent linked to a targeting compound via a saccharide residue, preferably a modified saccharide residue. Targeted glycoconjugates of the invention are generally described by the formula:
B-S-T
wherein

B is a bioactive agent;

S is a saccharide residue (e.g., a modified saccaharide residue); and

T is a targeting compound.

1. Preparing Targeted Glycoconjugates of the Invention

a. β-1,4-galactosyltransferase (GalT)

b. β-1,4-galactosyltransferase (GalT) catalyzes the transfer of galactose from the donor UDP-galactose, to an acceptor, N-acetylglucosamine (GlcNAc, present at the non-reducing terminal end of glycans of glycoproteins and glycolipids), to form a galactose-β-1,4-N-acetylglucosamine bond (Hill, UCLA Forum Med. Sci. 21:63-86 (1979). This reaction allows galactose to be linked to an N-acetylglucosamine that may itself be linked to a variety of other molecules, such as sugars and proteins, e.g., antibodies. In addition to GlcNAc as an acceptor, the enzyme can also use other sugars, such as N-acyl-substituted glucosamine and N-acetyl-D-mannosamine (Berliner, L. J. et al., Mol. Cell. Biochem., 62:37-42 (1984). The enzyme does not have an absolute requirement for the sugar donor UDP-Gal; instead, it exhibits polymorphic donor specificity, in that it also transfers glucose (Glc), D-deoxy-Glc, arabinose, GalNAc, and GlcNAc from their UDP derivatives (Berliner, L. J. and Robinson, R. D., Biochemistry, 21:6340-6343 (1982); Andree, P. J. and Berliner L. J., Biochim. Biophys. Acta. 544:489-495 (1982); Do, K. Y. et al., J. Biol. Chem., 270:18477-18451 (1995); Palcic, M. M and Hindsgaul, O., Glycobiology 1:205-209 (1991); Ramakrishnan, B. et al., J. Biol. Chem., 276:37665-37671 (2001)). This reaction can be used to generate many types of molecules, such as the glycoconjugates of the present invention, which have applications in research and medicine.

The present invention is based on the discovery that GalT tolerates alterations in its substrates/donors, such as UDP-galactose, and the ability to use unnatural substrates (altered donor specificity). In one embodiment, the catalytic domain of GalT has a tyrosine exchanged with another amino acid at an amino acid position corresponding to 289 in the bovine β(1,4)-galactosyltransferase I (see, for example, PCT/US2004/000470, filed Jan. 9, 2004, which is incorporated herein by reference). Examples of specific exchanges are Y289L, Y289I, and Y289N. The corresponding tyrosine in the human and mouse β(1,4)-galactosyltransferase I is located at amino acid position 285 and 286. Accordingly, those of skill in the art can readily determine equivalent amino acids in other β(1,4)-galactosyltransferase I catalytic domains and generate them through recombinant techniques known in the art. In one embodiment, a genetically engineered form of GalT, GalT (Y289L), is used to catalyze the formation of the glycoconjugates of the invention. The GalT (Y289L) has an enlarged binding pocket which enhances the catalytic activity toward GalNAc substrates without compromising specificity (See, Khidekel et al., 2003 and PCT/US04/00470, filed Jan. 9, 2004, both of which are incorporated herein by reference).

b. Modified Saccharide Compound (S)

In one approach, the glycoconjugates are constructed from their individual components, e.g., targeting compound (T), donor molecule including a saccharide residue (S), and bioactive agent (B). The donor molecule can include any of a series of UDP derivative compounds including carbon, hydrogen, and oxygen in which the atoms of the latter two elements are in the ratio of 2:1, especially those containing the group C₆H₁₀O₅, including fructose, glucose, D-glucose, sucrose, lactose, mannose, maltose, galactose, xylose, fucose, rhamnose and arabinose. In one embodiment, the donor molecule is a UDP derivative of galactose, glucose (Glc), D-deoxy-Glc, arabinose, GalNAc, and GlcNAc. In another one embodiment, the donor molecule includes a modified saccharide residue (S). In one embodiment, the saccharide is modified so as to include a functional group, such as amino (—NH₂), hydroxy (—OH), carboxyl (—COOH), thiol (—SH), phosphate, phosphinate, ketone, sulfate and sulfinate groups to aid in the attachment of the bioactive agent. For example, the modified saccharide (S) may include a ketone moiety which can be reacted with an amino group of a bioactive agent of interest so as to form a covalent bond between the two. Preferably, the functional group is one which is tolerated by the enzyme β-1,4-galactosyltransferase (GalT), or a mutant thereof, in that the enzyme is able to transfer the modified saccharide of the donor molecule to an acceptor molecule, e.g., a carbohydrate, glycoprotein, or glycolipid.

In one embodiment, the saccharide is modified so as to include a functional group at the C2 position of the saccharide ring, preferably a ketone functionality. In another embodiment, the modified saccharide is a galactose which is modified at the C2 position by the addition of ketone functionality (as described herein below).

c. Targeting Compound (T)

The targeting compound (T), which is discussed in detail herein below, is covalently bonded to a saccharide residue (S) with the use of a galactosyltranserfase enzyme, preferably β-1,4-galactosyltransferase (GalT). In one embodiment of the invention, a modified saccharide (S) is covalently associated with the targeting compound with the use of a genetically engineered GalT, such as Y289L GalT (as discussed above). The targeting compound can be any naturally occurring glycoprotein, glycolipid or carbohydrate or can be engineered, through chemical or recombinant techniques. For example, if the targeting compound does not include a GlcNAc residue, the compound can be engineered, either through recombinant or chemical techniques known in the art, so as to include such a residue. Preferably, the targeting compound includes an N-acetylglucosamine (GlcNAc) residue.

d. Bioactive Agent

The bioactive agent (B), which is discussed in detail herein below, is covalently associated with the saccharide residue (S) through a functional group present on the saccharide and/or the bioactive agent (as discussed above and below) or introduced thereon using one or more steps, e.g., oxidation reactions, reduction reactions, cleavage reactions and the like. The particular portion of the different components that are modified to provide for covalent linkage will be chosen so as not to substantially adversely interfere with that components desired binding and/or activity, e.g., for the bioactive agent, a region that does not affect the efficacy of the agent, such that a sufficient amount of the desired bioactive agent, e.g., drug, activity, is preserved.

The methods used to bind the bioactive agent (B) to the modified saccharide (S) depend on the structure of the bioactive agent. The bioactive compounds may preferably include a functional group which may be useful, for example, in forming covalent bonds with the saccharide residue, which are not generally critical for the activity of the bioactive agent. Examples of such functional groups include, for example, amino (—NH₂), hydroxy (—OH), carboxyl (—COOH), thiol (—SH), phosphate, phosphinate, ketone group, sulfate and sulfinate groups. If the bioactive compounds do not contain a useful group, one can be added to the bioactive compound by, for example, chemical synthetic means. Where necessary and/or desired, certain moieties on the components may be protected using blocking groups, as is known in the art, see, e.g., Green & Wuts, Protective Groups in Organic Synthesis (John Wiley & Sons) (1991).

Exemplary covalent bonds by which the bioactive compounds may be associated with the saccharide residue (S) include, for example, amide (—CONH—); thioamide (—CSNH—); ether (ROR′, where R and R′ may be the same or different and are other than hydrogen); ester (—COO—); thioester (—COS—); —O—; —S—; —Sₙ—, where n is greater than 1, preferably about 2 to about 8; carbamates; —NH—; —NR—, where R is alkyl, for example, alkyl of from about 1 to about 4 carbons; urethane; and substituted imidate; and combinations of two or more of these.

Covalent bonds between a bioactive agent (B) and a modified saccharide residue (S) may be achieved through the use of molecules that may act, for example, as spacers to increase the conformational and topographical flexibility of the compound. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like.

One of skill in the art can easily chose suitable compatible reactive groups for the bioactive agent and the modified saccharide, so as to generate a covalent bond between the bioactive agent and the modified saccharide. Also, while the glycoconjugates of the invention are generally described with the targeting agent as the acceptor molecule or structure onto which a donor molecule (e.g., UDP-galactose) is actively linked through the action of a catalytic domain of a galactosyltransferase, or mutant thereof, the bioactive agent (B) can also be an acceptor molecule. In this situation, the targeting compound (T) can be linked to the modified saccharide of the donor molecule via a functional chemical group well known in the art, e.g., a ketone group at the C2 position of galactose.

C. Bioactive Agents

A wide variety of bioactive agents (B) may be included in the compounds of the present invention, such as any biologically active, therapeutic or diagnostic compound/composition. In general, the term bioactive agent includes, but is not limited to: polypeptides, including proteins and peptides (e.g., insulin); releasing factors and releasing factor inhibitors, including Luteinizing Hormone Releasing Hormone (LHRH) and gonadotropin releasing hormone (GnRH) inhibitors; carbohydrates (e.g., heparin); nucleic acids; vaccines; and pharmacologically active agents such as anti-infectives such as antibiotics and antiviral agents; anti-fungal agents; analgesics and analgesic combinations; anesthetics; anorexics; anti-helminthics; anti-arthritic agents; respiratory drugs, including anti-asthmatic agents and drugs for preventing reactive airway disease; anticonvulsants; antidepressants; anti-diabetic agents; anti-diarrheals; anticonvulsants; antihistamines; anti-inflammatory agents; toxins, anti-migraine preparations; anti-nauseants; anticancer agents, including anti-neoplastic drugs; anti-parkinsonism drugs; anti-pruritics; anti-psychotics; antipyretics; antispasmodics; anticholinergics; sympathomimetics; xanthine derivatives; cardiovascular preparations including potassium and calcium channel blockers, beta-blockers, alpha-blockers, cardioprotective agents; anti-arrhythmics; anti-hyperlipidemic agents; anti-hypertensives; diuretics; anti-diuretics; receptor agonists, antagonists, and/or mixed function agonist/antagonists; vasodilators including general coronary, peripheral and cerebral; central nervous system stimulants; vasoconstrictors; cough and cold preparations, including decongestants; enzyme inhibitors; hormones such as estradiol, testosterone, progesterone and other steroids and derivatives and analogs, including corticosteroids; hypnotics; hormonolytics; immunosuppressive agents; muscle relaxants; parasympatholytics; central nervous system stimulants; diuretics; hypnoticsleukotriene inhibitors; mitotic inhibitors; muscle relaxants; genetic material, including nucleic acid, RNA, DNA, recombinant RNA, recombinant DNA, antisense RNA, antisense DNA, hammerhead RNA, a ribozyme, a hammerhead ribozyme, an antigene nucleic acid, a ribo-oligonucleotide, a deoxyribonucleotide, an antisense ribo-oligonucleotide, and/or an antisense deoxyribo-oligonucleotide; psychostimulants; sedatives; anabolic agents; vitamins; herbal remedies; anti-metabolic agents; anxiolytics; attention deficit disorder (ADD) and attention deficit hyperactivity disorder (ADHD) drugs; neuroleptics; and tranquilizers.

Specific examples of bioactive agents (B) include, but are not limited to, the following:

analgesic agents—hydrocodone, hydromorphone, levorphanol, buprenorphine, butorphanol, oxycodone, oxymorphone, codeine, morphine, nalbuphine, butethamine, fenalcomine, hydroxytetracaine, naepaine, orthocaine, piridocaine, salicyl alcohol, alfentanil, fentanyl, meperidine and sufentanil, diphenylheptanes such as levomethadyl, methadone and propoxyphene, and anilidopiperidines such as remifentanil;

antiandrogens—bicalutamide, flutamide, hydroxyflutamide, zanoterine and nilutamide;

anxiolytic agents and tranquilizers—diazepam, alprazolam, chlordiazepoxide, clonazepam, halazepam, lorazepam, oxazepam and clorazepate;

anti-arthritic agents—hydroxychloroquine, gold-based compounds such as auranofin, aurothioglucose and gold thiomalate, and COX-2 inhibitors such as celecoxib and rofecoxib;

antibiotics (including anti-neoplastic antibiotics)—vancomycin, bleomycin, pentostatin, mitoxantrone, mitomycin, dactinomycin, plicamycin and amikacin;

antibacterial agents—2-p-sulfanilyanilinoethanol, 4,4′-sulfinyldianiline, 4 sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefmenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, prinycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin and vancomycin;

anticancer agents, including antineoplastic agents and cytotoxic drugs—such as alkylating agents, anti-proliferative agents, tubulin binding agents and the like, the anthracycline family of drugs, the vinca drugs, the mitomycins, the bleomycins, the cytotoxic nucleosides, the pteridine family of drugs, diynenes, paclitaxel, docetaxel, camptothecin and its analogues and derivatives (e.g., 9-aminocamptothecin, 9-nitrocamptothecin, 10-hydroxy-camptothecin, irinotecan, adriamycin, daunorubicin, methotrexate, methopterin, dichloromethotrexate, mitomycin C, porfiromycin, 5-fluorouracil, 6-mercaptopurine, aminopterin, cytosine arabinoside, caminomycin, topotecan, 20-O-glucopyranosyl camptothecin), taxanes (baccatins, cephalomannine and their derivatives), carboplatin, cisplatin, interferon-2A, interferon-2B, interferon-N3 and other agents of the interferon family, 6-azauridine, 6-diazo-5-oxo-L-norleucine, aclacinomycin(s), ancitabine, azacitadine, azaserine, capecitabine, carubicin, carzinophillin A, chlorozotocin, chromomycin(s), cytarabine, denopterin, doxifluridine, doxorubicin, edatrexate, eflornithine, elliptinium, enocitabine, epirubicin, floxuridine, fludarabine, idarubicin, mannomustine, menogaril, mitobronitol, mitolactol, mitoxantrone, mopidamol, mycophenolic acid, nogalamycin, olivomycin(s), pentostatin, peplomycin, pirarubicin, piritrexim, plicamycin, podophyllinic acid 2-ethylhydrazine, prednimustine, pteropterin, puromycin, ranimustine, streptonigrin, streptozocin, thiamiprine, thioguanine, N-[[5-[[(1,4-Dihydro-2-methyl-4oxo-6-quinazolinyl)methyl]methylamino]-2-thienyl]carbonyl]-L-glutamic acid, toptecan, trimetrexate, tubercidin, ubenimex, zorubicin, levamisole, altretamine, cladribine, bovine-calmette-guerin (BCG), aldesleukin, tretinoin, procarbazine, dacarbazine, gemcitabine, mitotane, asparaginase, porfimer, mesna, amifostine, mitotic inhibitors including podophyllotoxin, or podophyllotoxin derivatives such as tenipside, etoposide or etoposide phosphate, melphalan, leurosidine, vindesine, leurosine and vinca alkaloids such as vinorelbine, vincristine and vinblastine;

antidepressant drugs—selective serotonin reuptake inhibitors such as sertraline, paroxetine, fluoxetine, fluvoxamine, citalopram, venlafaxine and nefazodone; tricyclic anti-depressants such as amitriptyline, doxepin, nortriptyline, imipramine, trimipramine, amoxapine, desipramine, protriptyline, clomipramine, mirtazapine and maprotiline; other anti-depressants such as trazodone, buspirone and bupropion;

anti-estrogens—tamoxifen, clomiphene and raloxifene;

anti-fungals—amphotericin B, azaserine, candicidlin(s), chlorphenesin, dermostatin(s), filipin, fungichromin, mepartricin, nystatin, oligomycin(s), perimycin A, tubercidin, imidazoles, triazoles, and griesofulvin;

anti-hyperlipidemic agents—HMG-CoA reductase inhibitors such as atorastatin, simvastatin, pravastatin, lovastatin and cerivastatin sodium, and other lipid-lowering agents such as clofibrate, fenofibrate, gemfibrozil and tacrine;

anti-metabolic agents—methotrexate, fluorouracil, floxuridine, cytarabine, mercaptopurine and fludarabine phosphate;

anti-migraine preparations-zolmitriptan, naratriptan, sumatriptan, rizatriptan, methysergide, ergot alkaloids and isometheptene;

anti-psychotic agents—chlorpromazine, prochlorperazine, trifluoperazine, promethazine, promazine, thioridazine, mesoridazine, perphenazine, acetophenazine, clozapine, fluphenazine, chlorprothixene, thiothixene, haloperidol, droperidol, molindone, loxapine, risperidone, pimozide and domepezil;

anti-thrombotic agents—including argatroban, coumetarol, dicoumarol, ethyl biscoumacetate, ethylidene dicoumarol, iloprost, lamifiban, taprostene, tioclomarol and tirofiban;

aromatase inhibitors—anastrozole and letrozole;

attention deficit disorder and attention deficit hyperactivity disorder drugs—methylphenidate and pemoline;

cardiovascular preparations—angiotensin converting enzyme (ACE) inhibitors; diuretics; pre, and after-load reducers; iloprost; cardiac glycosides such as digoxin and digitoxin; inotropes such as aminone and milrinone; calcium channel blockers such as verapamil, nifedipine, nicardipene, felodipine, isradipine, nimodipine, bepridil, amlodipine and diltiazem; beta-blockers such as pindolol, propafenone, propranolol, esmolol, sotalol and acebutolol; antiarrhythmics such as moricizine, ibutilide, procainamide, quinidine, disopyramide, lidocaine, phenyloin, tocamide, mexiletine, flecamide, encamide, bretylium and amiodarone; cardioprotective agents such as dexrazoxane and leucovorin;

GnRH inhibitors and other hormonolytics and hormones—leuprolide, goserelin, chlorotrianisene, dinestrol and diethylstilbestrol;

herbal remedies—such as melatonin;

immunosuppressive agents—6-mercaptopurine, amiprilose, bucillamine, gusperimus, mycophenolic acid, procodazole, romurtide, sirolimus (rapamycin), tacrolimus, ubenimex, 6-thioguanine, 6-aza-guanine, azathiopurine, cyclosporin and methotrexate;

lipid-soluble vitamins-tocopherols and retinols;

leukotriene inhibitors-zafirlukast, zileuton and montelukast sodium; nonsteroidal anti-inflammatory drugs (NSAIDs)—diclofenac, 3-amino-4-hydroxybutyric acid, aceclofenac, ahninoprofen, amfenac, bromosaligenin, bumadizon, carprofen, diflunisal, ditazol, enfenamic acid, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine or tolfenamic acid, flurbiprofen, ibuprofen, ketoprofen, piroxicam, naproxen, indomethacin, sulindac, tolmetin, meclofenamate, mefenamic acid, etodolac, ketorolac and bromfenac;

peptide drugs—leuprolide, somatostatin, oxytocin, calcitonin and insulin;

peripheral vascular dilator agents—cyclandelate, isoxsuprine and papaverine;

respiratory drugs—such as theophylline, oxytriphylline, aminophylline and other xanthine derivatives;

toxins—including diphtheria toxin, prutusis toxin, botulinum toxin, tetanus toxin, anthrax toxin; toxins from venomous snakes, ricin, abrin, ribonuclease RNase, DNase I, Staphylococcal enterotoxin-A, pokeweed antiviral protein, gelonin, pertussis toxin, Pseudomonas exotoxin, Pseudomonas endotoxin, and genetically engineered toxins, including human α-lactalbumin made lethal to tumor cells (HAMLET (a complex of human α-lactalbumin and oleic acid (C18:1:9 cis) that kills tumor cells; Svensson et al., Protein Science, 12:2794-2804 (2003));

steroids—progestogens such as fluorogestone acetate, hydroxyprogesterone, hydroxyprogesterone acetate, hydroxyprogesterone caproate, medroxyprogesterone acetate, megestrol, norethindrone, norethindrone acetate, norethisterone, norethynodrel, desogestrel, 3-keto desogestrel, gestadene and levonorgestrel; estrogens such as estradiol and its esters (e.g., estradiol benzoate, valerate, cyprionate, decanoate and acetate), ethynyl estradiol, estriol, estrone, mestranol and polyestradiol phosphate; corticosteroids such as betamethasone, betamethasone acetate, cortisone, hydrocortisone, hydrocortisone acetate, corticosterone, fluocinolone acetonide, flunisolide, fluticasone, prednisolone, prednisone and triamcinolone; androgens and anabolic agents such as aldosterone, androsterone, testosterone and methyl testosterone;

topoisomerase inhibitors—camptothecin, anthraquinones, anthracyclines, temiposide, etoposide, topotecan and irinotecan;

immunosuppressive agents such as cyclophosphamides as exemplified by cyclosporin-A, mycophenolic acid, rapamycin, 6-mercaptopurine, azothioprine, prednisone, prednisolone, cortisone, azidothymide and OKT-3;

genetic materials—such as genes which code growth factors and other proteins such as vascular endothelial growth factor, fibroblast growth factor, BCl-2, cystic fibrosis transmembrane regulator, nerve growth factor, human growth factor, erythropoietin, tumor necrosis factor, and interleukin-2, histocompatibility genes such as HLA-B7, genes coding for enzymes regulating metabolism such as glycolytic enzymes, enzymes of the citric acid cycles and oxidative phosphorylation, genes for hormones such as insulin, glucagon and vasopressin, oncogenes and proto-oncogenes such as c-fos and c-jun, tumor suppression factors such as p53 and telomeres.

Additional examples of “bioactive agents” (B) include, but are not limited to, Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11 (“IL-11”), Interleukin-12 (“IL-12”), Interleukin-13 (“IL-13”), Interleukin-15 (“IL-15”), Interleukin-16 (“IL-16”), Interleukin-17 (“IL-17”), Interleukin-18 (“IL-18”), lipid A, phospholipase A2, endotoxins, staphylococcal enterotoxin B and other toxins, Type I Interferon, Type II Interferon, Tumor Necrosis Factor (“TNFα”), Transforming Growth Factor-β(“TGFβ”), Lymphotoxin, Migration Inhibition Factor, Granulocyte-Macrophage Colony-Stimulating Factor (“CSF”), Monocyte-Macrophage CSF, Granulocyte CSF, vascular epithelial growth factor (“VEGF”), Angiogenin, transforming growth factor (“TGFα”), heat shock proteins, carbohydrate moieties of blood groups, Rh factors, fibroblast growth factor, hormones, such as growth hormone, insulin, glucogen, parathyroid hormone, leutinizing hormone, follicle stimulating hormone, and leutinizing hormone releasing hormone, cell surface receptors, antibodies, chemotherapeutic agents, and other inflammatory and immune regulatory proteins, nucleotides, DNA, RNA, sense, antisense, cancer cell specific antigens, such as MART, MAGE, BAGE, and HSPs; and immunotherapy drugs, such as AZT.

D. Targeting Compound (T)

A wide variety of targeting compounds (T) may be employed in the present glycoconjugate compounds depending, for example, on the particular tissue, cell or receptor to be targeted. Generally speaking, materials which may be employed as targeting compounds (T) include, for example, peptides or proteins such as antibodies, including monoclonal and polyclonal (e.g., anti-CD20 antibody, anti-IL-2Rα antibody, anti-B-FN antibody) and fragments thereof, ligands, including receptor ligands/proteins (preferably those that specifically bind to their receptors), peptides, polypeptides (e.g., Type I interferon, Type II interferon), cytokines (e.g., interleukin-1 (“IL-1”), interleukin-2 (“IL-2”), interleukin-3 (“IL-3”), interleukin-4 (“IL-4”), interleukin-5 (“L-5”), interleukin-6 (“IL-6”), Interleukin-7 (“IL-7”), interleukin-8 (“IL-8”), Interleukin-10 (“IL-10”), Interleukin-11 (“IL-11”), interleukin-12 (“IL-12”), interleukin-13 (“IL-13”) and tumor necrosis factor (“TNFα”)), growth factors (e.g., epidermal growth factor (EGF), transforming growth factor-β(“TGF-β”), vascular epithelial growth factor (“VEGF”), transforming growth factor-alpha (“TGFα”)) or fragments thereof, vitamins and vitamin analogues such as folate, vitamin-B12, vitamin B6, niacin, nicotinamide, vitamin A and retinoid derivatives, ferritin and vitamin D, sugar molecules (e.g., glucose and glycogen) and polysaccharides, glycopeptides and glycoproteins, phospholipids, steroids, steroid analogs, hormones, cofactors, bioactive agents, and genetic material, including nucleosides, nucleotides and polynucleotides and drug molecules such as cyclosporin-A, prostaglandin and prostacyclin.

An embodiment of the present invention provides a glycoconjugate in which one or more bioactive agents are bound to a modified saccharide residue, e.g., a modified galactose, which is in turn bound to a targeting compound, e.g., a compound capable of binding a receptor on a cell membrane. In this manner, many targeting glycoconjugates can be constructed. For example, a gene delivery system for genetic therapy can be produced by binding a nucleotide and a ligand or antibody to the modified sugar. A therapeutic compound for cancer can be produced by binding a chemotherapeutic agent and a ligand or antibody, e.g., an antibody to a cancer antigen, to the modified sugar residue.

Further examples include the simultaneous binding of a cancer cell marker, such as MART and a chemotherapeutic agent, such as methotrexate, to the sugar residue. Another example is binding of IL-2 and an anti-viral compound for the treatment of virally infected T-cells in AIDS patients.

Reverse targeting is also within the scope of the invention. As used herein, “reverse targeting” refers to the attraction of target cells to the bioactive agent/device via chemotaxis (Kmamoto et al., Nat. Biotechnol., 20:64 (2002).

1. Antibodies

In one embodiment, the targeting compound is an antibody or a fragment thereof. As used herein, the term “antibody” (Ab) or “monoclonal antibody” (Mab) is meant to include intact molecules as well as antibody portions (e.g., Fab and F(ab)₂ portions and Fv fragments) which are capable of specifically binding to a cell surface marker. Such portions are typically produced by proteolytic cleavage, using enzymes such as papain (to produce Fab portions) or pepsin (to produce F(ab′)₂ portions). Alternatively, antigen-binding portions can be produced through the application of recombinant DNA technology.

The immunoglobulin can be a “chimeric antibody” as that term is recognized in the art. Also, the immunoglobulin may be a “bifunctional” or “hybrid” antibody, that is, an antibody which may have one arm having a specificity for one antigenic site, such as a tumor associated antigen, while the other arm recognizes a different target, for example, a hapten which is, or to which is bound, an agent lethal to the antigen-bearing tumor cell. Alternatively, the bifunctional antibody may be one in which each arm has specificity for a different epitope of a tumor associated antigen of the cell to be therapeutically or biologically modified. In any case, the hybrid antibodies have a dual specificity, preferably with one or more binding sites specific for the hapten of choice or one or more binding sites specific for a target antigen, for example, an antigen associated with a tumor, an infectious organism, or other disease state.

Biological bifunctional antibodies are described, for example, in European Patent Publication, EPA 0 105 360, which is incorporated herein by reference. Hybrid or bifunctional antibodies may be derived biologically, by cell fusion techniques, or chemically, especially with cross-linking agents or disulfide bridge-forming reagents, and may be comprised of those antibodies and/or fragments thereof. Methods for obtaining such hybrid antibodies are disclosed, for example, in PCT application WO83/03679, published Oct. 27, 1983, and published European Application EPA 0217577, published Apr. 8, 1987, which are incorporated herein by reference. In one embodiment, the bifunctional antibodies are biologically prepared from a “polydome” or “quadroma” or are synthetically prepared with cross-linking agents such as bis-(maleimideo)-methyl ether (“BMME”), or with other cross-linking agents familiar to those skilled in the art.

In addition, the immunoglobin may be a single chain antibody (“SCA”). These may consist of single chain Fv fragments (“scFv”) in which the variable light (“V[L]”) and variable heavy (“V[H]”) domains are linked by a peptide bridge or by disulfide bonds. Also, the immunoglobulin may consist of single V[H]domains (dAbs) which possess antigen-binding activity. See, e.g., G. Winter and C. Milstein, Natures 349:295 (1991); R. Glockshuber et al., Biochemistry, 29:1362 (1990); and, E. S. Ward et al., Nature, 341:544 (1989).

In one embodiment of the present invention, the antibodies are chimeric monoclonal antibodies. As used herein, the term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Other forms of chimeric antibodies encompassed by the invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques well known in the art. See, e.g., Morrison, S. L. et al., Proc. Nat'l Acad. Sci., 81:6851 (1984).

Encompassed by the term “chimeric antibody” is the concept of “humanized antibody,” that is those antibodies in which the framework or “complementarity” determining regions (“CDR”) have been modified to comprise the CDR of an immunoglobulin of different specificity as compared to that of the parent immunoglobulin. (See, e.g., EPA 0 239 400 (published Sep. 30, 1987)) In a preferred embodiment, a murine CDR is grafted into the framework region of a human antibody to prepare the “humanized antibody.” See, e.g., L. Riechmann et al., Nature. 332:323 (1988); M. S. Neuberger et al., Nature, 314:268 (1985).

Furthermore, the immunoglobulin (antibody), or fragment thereof, used in the present invention may be polyclonal or monoclonal in nature. Monoclonal antibodies are the preferred immunoglobulins. The preparation of such polyclonal or monoclonal antibodies is well known to those skilled in the art. See, e.g., G. Kohler and C. Milstein, Nature, 256:495 (1975). The antibodies of the present invention may be prepared by any of a variety of methods. For example, cells expressing the cell surface marker or an antigenic portion thereof can be administered to an animal in order to induce the production of sera containing polyclonal antibodies. In a preferred method, a preparation of protein is prepared and purified so as to render it substantially free of natural contaminants. Such a preparation is then introduced into an animal in order to produce polyclonal antisera of greater specific activity. However, the present invention should not be construed as limited in scope by any particular method of production of an antibody whether bifunctional, chimeric, bifunctional-chimeric, humanized, or an antigen-recognizing fragment or derivative thereof.

In a preferred embodiment, the antibodies of the present invention are monoclonal antibodies (or portions thereof). Such monoclonal antibodies can be prepared using hybridoma technology (Kohler et al., Nature, 256:495 (1975); Kohler et al., Eur. J. Immunol., 6:511 (1976); Kohler et al, Eur. J. Immunol., 6:292 (1976); Hammerling et al., In: “Monoclonal Antibodies and T-Cell Hybridomas,” Elsevier, N.Y., pp. 563-681 (1981)). In general, such procedures involve immunizing an animal (preferably a mouse) with a protein antigen or with a protein-expressing cell (suitable cells can be recognized by their capacity to bind antibody). The splenocytes of such immunized mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described by Wands et al., Gastroenterology, 80:225-232 (1981). The hybridoma cells obtained through such a selection are then assayed to identify clones which secrete antibodies capable of binding the antigen. In addition, hybridomas and/or monoclonal antibodies which are produced by such hybridomas and which are useful in the practice of the present invention are publicly available from sources such as the American Type Culture Collection (“ATCC”) 10801 University Boulevard, Manassas, Va. 20110-2209 or, commercially, for example, from Boehringer-Mannheim Biochemicals, P.O. Box 50816, Indianapolis, Ind. 46250. Myeloma cell lines are also publicly available from, for example, the American Type Culture Collection.

The antibodies of the present invention may be labeled, for example, for detection or diagnostic purposes, e.g., imaging. Labels for the antibodies of the present invention include, but are not limited to, the following:

examples of enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase;

examples of radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷CU, ²¹⁷Ci, ²¹¹At, ²¹²Pb, ⁴⁷Sc, and ¹⁰⁹Pd;

examples of suitable non-radioactive isotopic labels include ¹⁵⁷Gd, ⁵⁵Mn, ¹⁶²Dy, ⁵²Tr, and ⁵⁶Fe;

examples of fluorescent labels include an ¹⁵²Eu label, a fluorescein label, an isothiocyanate label, a rhodamine label, a phycoerythrin label, aphycocyanin label, an allophycocyanin label, an o-phthaldehyde label, and a fluorescamine label;

examples of toxin labels include diphtheria toxin, ricin, and cholera toxin;

examples of chemiluminescent labels include a luminal label, an isoluminal label, an aromatic acridinium ester label, an imidazole label, an acridinium salt label, an oxalate ester label, a luciferin label, a luciferase label, and an aequorin label; and

examples of nuclear magnetic resonance contrasting agents include heavy metal nuclei such as Gd, Mn, and Fe.

Typical techniques for binding the above-described labels to antibodies are provided by Kennedy et al., Clin. Chim. Acta. 70:1-31 (1976), and Schurs et al., Clin. Chim. Acta, 81:1-40 (1977), which are incorporated by reference herein.

In one embodiment, the glycoconjugates of the invention include monoclonal antibodies, such as those directed against tumor antigens, for use as cancer therapeutics. Generally, monoclonal antibodies have one N-linked bi-antennary oligosaccharide attached at the IgG-Fc region. The terminal sugars of the oligosaccharide moiety comes in several glycoforms, for example, some are desialated, degalactosylated, with only terminal N-acetylglucosaminyl residues. The monoclonal antibodies carrying only terminal N-acetylgucosamine on the bi-antennary oligosaccharide moieties, the G₀ glycoform, can be generated by de-sialylation and de-galactosylation of the monoclonal antibodies. With the mutant Tyr289Leu-Gal-T1 (Y289L GalT1)and UDP-α-galactose-C-2-modified, a galactose moiety that has a chemically reactive group attached at the C2 position of galactose, can then be transferred to Go glycoform of the monoclonal antibody. The chemically reactive group can include, for example, a ketone moiety that can serve as a neutral, yet versatile chemical handle to add other agents, such as bioactive agents, to the compound.

E. Testing Glycoconjugates of the Present Invention

The resulting glycoconjugates are then screened for those conjugates that exhibit the desired effect, e.g., targeted bioactive agent delivery. Any convenient screening assay may be employed. Typically, the screening assay will involve observing the distribution of the glycoconjugate and comparing it to a free bioactive agent control, e.g., in a suitable cell and/or animal model. As such, one can administer labeled glycoconjugates of the invention to a test animal and then observe its distribution in the animal at one or more periods following administration of the glycoconjugate. By comparing the observed results to those obtained with a control, the distribution of the glycoconjugate can be evaluated with respect to whether it is targeted to a specific cell/tissue type as compared to a free bioactive agent control. Other assays may also be employed.

F. Therapeutic Uses

The present invention comprises a compound, specifically a glycoconjugate, and method for administering bioactive agents in a targeted manner to an organism, e.g., a human or animal. Generally, the compound according to the present invention comprises a bioactive agent linked to a compound which has an affinity for a target cell (a targeting compound), for example, an antibody or antibody fragment which is specific to, for example, a cancer cell, by a sugar residue. Preferably, the sugar residue is a modified sugar residue. More preferably, the sugar residue is a modified galactose. Preferably, the galacatose is modified at the C2 position in a manner in which the C2 position includes a ketone group.

The glycoconjugates of the invention can be used to treat and/or diagnose a variety of diseases and/or disorders afflicting an organism. Due to the targeted nature of the therapy, smaller doses of the bioactive agent may be used than in conventional therapy. In one embodiment, the glycoconjugates of the invention are used for specific, targeted delivery of bioactive agents, including toxic drugs (e.g., toxins, radionuclides), to therapeutically-relevant tissues/cells of the body, e.g., tumors. In another embodiment of the invention, the glycoconjugates of the invention are used to deliver bioactive agents, including DNA vectors, to therapeutically-relevant cells for genetic corrections. In another embodiment, the glycoconjugates of the invention are used to deliver bioactive agents, such as those which specifically target the vasculature, as a cancer treatment in which the targeting agent targets neovasculature forming around tumors (Halin et al., Nat. Biotechnol., 20:264 (2002)), or in pulmonary, cardiovascular, and inflammatory diseases. In yet another embodiment, the glycoconjugates of the invention are used to deliver bioactive agents to targeted pathogen-infected cells (infected cells generally undergo changes in cell-surface molecule expression, thereby allowing one to target those cells expressing the altered cell-surface molecule expression).

As further examples, the glycoconjugates of the invention are useful for the treatment of a number of diseases and/or disorders including, but not limited to:

cancer, both solid tumors as well as blood-borne cancers, such as leukemia;

hyperproliferative disorders that can be treated by the compounds of the invention include, but are not limited to, neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Similarly, other hyperproliferative disorders can also be treated by the glycoconjugates of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease/disorder;

hormone deficiency diseases, such as growth hormone deficiency disease and osteoporosis;

hormone abnormalities due to hypersecretion, such as acromegaly;

infectious diseases, such as septic shock, or those caused by viruses, including but not limited to, DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia;

bacterial or fungal infections that can cause disease or symptoms and that can be treated by the glycoconjugates of the invention include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Emphysema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, and wound infections;

parasitic infections causing disease or symptoms that can be treated by the glycoconjugates of the invention include, but are not limited to, the following families: amebiasis, babesiosis, coccidiosis, cryptosporidiosis, dientamoebiasis, dourine, ectoparasitic, giardiasis, hehninthiasis, leishmaniasis, theileriasis, toxoplasmosis, trypanosomiasis, and trichomonas;

cardiovascular diseases and disorders, including dysfunctional conditions of the heart, arteries, and veins that supply oxygen to vital life-sustaining areas of the body like the brain, the heart itself, and other vital organs. In other words, cardiovascular diseases and disorders are diseases and disorders which affect the proper functioning of the heart and blood vessels, including, but limited to, myocardial infarction (heart attack), cerebrovascular diseases (stroke), transient ischaemic attacks (TIA), peripheral vascular diseases, arteriosclerosis, angina, high blood pressure, high cholesterol, arrhythmia;

genetic diseases, such as enzyme deficiency diseases (e.g., inability to metabolize phenylalanine resulting in phenylketanuria);

autoimmune diseases which may be treated using the glycoconjugates of the present invention include, but are not limited to Addison's Disease, hemolytic anemia, antiphospholipid syndrome, rheumatoid arthritis, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, polyendocrinopathies, purpura, Reiter's Disease, Stiff-Man Syndrome, autoimmune thyroiditis, systemic lupus erythematosus, autoimmune pulmonary inflammation, Guillain-Barre Syndrome, insulin dependent diabetes mellitis, autoimmune inflammatory eye disease, autoimmune hemolysis, psoriasis, juvenile diabetes, primary idiopathic myxedema, autoimmune asthma, scleroderma, chronic hepatitis, hypogonadism, pernicious anemia, vitiligo, alopecia greata, Coeliac disease, autoimmune enteropathy syndrome, idiopathic thrombocytic purpura, acquired splenic atrophy, idiopathic diabetes insipidus, infertility due to antispermatazoan antibodies, sudden hearing loss, sensoneural hearing loss, polymyositis, autoimmune demyelinating diseases, traverse myelitis, ataxic sclerosis, progressive systemic sclerosis, dermatomyositis, polyarteritis nodosa, idiopathic facial paralysis, cryoglobulinemia, inflammatory bowel diseases, Hashimoto's disease, adrenalitis, hypoparathyroidism, and ulcerative colitis;

allergic reactions and conditions, such as asthma (particularly allergic asthma) or other respiratory problems;

organ rejection or graft-versus-host disease (GVHD); and

immune deficiency diseases, such as AIDS.

Thus, the glycoconjugates of the present invention find use in the pharmacological treatment of a host of conditions/disorders. In the methods of the invention, an effective amount of the glycoconjugate is administered to an organism.

As discussed below, the composition of the present invention can also be used to vaccinate a human or animal against bioactive agents.

1. Vaccine

One embodiment of the invention provides methods for invoking an immune response in a mammal such as a human, including vaccinating a mammal with a compound or composition described herein. Therefore, one embodiment of the present invention is to use the glycoconjugates described herein as a vaccine preparation.

The vaccine against the glycoconjugates may be prepared by any method known in the art. For example, glycoconjugates of the present invention are prepared and are then injected into an appropriate animal. The compositions according to the present invention may be administered in a single dose or they may be administered in multiple doses, spaced over a suitable time scale to fully utilize the secondary immunization response. For example, antibody titers may be maintained by administering boosters once a month. The vaccine may further comprise a pharmaceutically acceptable adjuvant, including, but not limited to Freund's complete adjuvant, Freund's incomplete adjuvant, lipopolysaccharide, monophosphoryl lipid A, muramyl dipeptide, liposomes containing lipid A, alum, muramyl tripeptide-phosphatidylethanoloamine, keyhole and limpet hemocyanin.

The glycoconjugates of the invention are useful for raising an immune response and treating hyperproliferative disorders. Examples of hyperproliferative disorders that can be treated by the compounds of the invention include, but are not limited to, neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated by the glycoconjugates of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The glycoconjugates of the present invention are also useful for raising an immune response against infectious agents. Viruses are one example of an infectious agent that can cause disease or symptoms that can be treated by the compounds of the invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia.

Similarly, bacterial or fungal agents that can cause disease or symptoms and that can be treated by the glycoconjugates of the invention include, but are not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Emphysema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, and wound infections.

Moreover, parasitic agents causing disease or symptoms that can be treated by the glycoconjugates of the invention include, but are not limited to, the following families: amebiasis, babesiosis, coccidiosis, cryptosporidiosis, dientamoebiasis, dourine, ectoparasitic, giardiasis, helminthiasis, leishmaniasis, theileriasis, toxoplasmosis, trypanosomiasis, and trichomonas.

Additionally, the glycoconjugates of the invention are useful for treating autoimmune diseases. An autoimmune disease is characterized by the attack by the immune system on the tissues of the victim. In autoimmune diseases, the recognition of tissues as “self” apparently does not occur, and the tissue of the afflicted subject is treated as an invader—i.e., the immune system sets about destroying this presumed foreign target. The compounds of the present invention are therefore useful for treating autoimmune diseases by desensitizing the immune system to these self antigens by provided a TCR signal to T cells without a costimulatory signal or with an inhibitory signal.

Examples of autoimmune diseases which may be treated using the glycoconjugates of the present invention include, but are not limited to Addison's Disease, hemolytic anemia, antiphospholipid syndrome, rheumatoid arthritis, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, polyendocrinopathies, purpura, Reiter's Disease, Stiff-Man Syndrome, autoimmune thyroiditis, systemic lupus erythematosus, autoimmune pulmonary inflammation, Guillain-Barre Syndrome, insulin dependent diabetes mellitis, autoimmune inflammatory eye disease, autoimmune hemolysis, psoriasis, juvenile diabetes, primary idiopathic myxedema, autoimmune asthma, scleroderma, chronic hepatitis, hypogonadism, pernicious anemia, vitiligo, alopecia greata, Coeliac disease, autoimmune enteropathy syndrome, idiopathic thrombocytic purpura, acquired splenic atrophy, idiopathic diabetes insipidus, infertility due to antispermatazoan antibodies, sudden hearing loss, sensoneural hearing loss, polymyositis, autoimmune demyelinating diseases, traverse myelitis, ataxic sclerosis, progressive systemic sclerosis, dermatomyositis, polyarteritis nodosa, idiopathic facial paralysis, cryoglobulinemia, inflammatory bowel diseases, Hashimoto's disease, adrenalitis, hypoparathyroidism, and ulcerative colitis.

Similarly, allergic reactions and conditions, such as asthma (particularly allergic asthma) or other respiratory problems, may also be treated by glycoconjugates of the invention. Moreover, the glycoconjugates of the invention can be used to treat anaphylaxis, hypersensitivity to an antigenic molecule, or blood group incompatibility.

The glycoconjugates of the invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of the glycoconjugates of the invention that inhibit an immune response may be an effective therapy in preventing organ rejection or GVHD.

The glycoconjugates of the invention which can inhibit an immune response are also useful for treating and/or preventing atherosclerosis; olitis; regional enteritis; adult respiratory distress syndrome; local manifestations of drug reactions, such as dermatitis, etc.; inflammation-associated or allergic reaction patterns of the skin; atopic dermatitis and infantile eczema; contact dermatitis; psoriasis; lichen planus; allergic enteropathies; allergic rhinitis; bronchial asthma; hypersensitivity or destructive responses to infectious agents; poststreptococcal diseases, e.g. cardiac manifestations of rheumatic fever, and the like.

G. Manufacture and Storage

Standard techniques and reagents known to those skilled in the art of pharmaceutical formulation and drug delivery may be employed in connection with the preparation of the present compositions. Techniques that may be suitable are described, for example, in Remington: The Science and Practice of Pharmacy, 19^{th} Ed. (Easton, Pa.: Mack Publishing Co., 1995), the disclosure of which is incorporated herein by reference. Remington's discloses, inter alia, conventional methods of preparing pharmaceutical compositions that may be used as described or modified to prepare compositions as described herein.

The compositions of the invention ordinarily will be stored in unit or multi-dose containers, for example, sealed ampules or vials, as an aqueous solution or as a lyophilized formulation for reconstitution.

H. Pharmaceutical Preparations. Administration and Kits

The pharmaceutical compositions of the present invention may be administered by any means that results in the contact of the bioactive agent with the agent's site or site(s) of action on or in an organism, e.g., a patient. The compositions may be administered by any conventional means available for use in conjunction with pharmaceuticals, either as individual therapeutic agents or in a combination of therapeutic agents. For example, the present pharmaceutical compositions may be administered alone, or they may be used in combination with other therapeutically active ingredients.

The targeted therapeutics, meaning the targeted glycoconjugates produced according to the present invention, can be administered to a mammalian host by any route. Thus, as appropriate, administration can be orally, intravenously, rectally, parenterally, intracistemally, intradermally, intravaginally, intraperitoneally, topically (as by powders, ointments, gels, creams, drops or transdermal patch), bucally, or as an oral or nasal spray. The term “parenteral” as used herein refers to modes of administration which include intravenous, intramuscular, intraperitoneal, intrasternal, subcutaneous and intraarticular injection and infusion. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

In addition, administration can be by periodic injections of a bolus of the therapeutic or can be made more continuous by intravenous or intraperitoneal administration from a reservoir which is external (e.g., an i.v. bag). In certain embodiments, the therapeutics of the instant invention can be pharmaceutical-grade. That is, certain embodiments comply with standards of purity and quality control required for administration to humans. Veterinary applications are also within the intended meaning as used herein.

The formulations, both for veterinary and for human medical use, of the therapeutics according to the present invention typically include such therapeutics in association with a pharmaceutically acceptable carrier therefor and optionally other ingredient(s). The carrier(s) can be “acceptable” in the sense of being compatible with the other ingredients of the formulations and not deleterious to the recipient thereof. Pharmaceutically acceptable carriers, in this regard, are intended to include any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional media or agent is incompatible with the glycoconjugate (or components thereof, e.g., the bioactive agent (B), the saccharide residue (S) or the targeting compound (T)), use thereof in the compositions is contemplated. The formulations can conveniently be presented in dosage unit form and can be prepared by any of the methods well known in the art.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences. Formulations for parenteral administration also can include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, hydrogenated naphthalenes, and the like. Formulations for direct administration can include glycerol and other compositions of high viscosity. Other potentially useful parenteral carriers for these therapeutics include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of discrete units such as capsules, gelatin capsules, sachets, tablets, troches, or lozenges, each containing a predetermined amount of the drug; in the form of a powder or granules; in the form of a solution or a suspension in an aqueous liquid or non-aqueous liquid; or in the form of an oil-in-water emulsion or a water-in-oil emulsion. The therapeutic can also be administered in the form of a bolus, electuary or paste. A tablet can be made by compressing or molding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Molded tablets can be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition can be sterile and can be fluid to the extent that easy syringability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by sterilization, e.g., filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient.

Formulations suitable for topical administration, including eye treatment, include liquid or semi-liquid preparations such as liniments, lotions, gels, applicants, oil-in-water or water-in-oil emulsions such as creams, ointments or pasts; or solutions or suspensions such as drops. Formulations for topical administration to the skin surface can be prepared by dispersing the therapeutic with a dermatologically acceptable carrier such as a lotion, cream, ointment or soap. In some embodiments, useful are carriers capable of forming a film or layer over the skin to localize application and inhibit removal.

For inhalation treatments, such as for asthma, inhalation of powder (self-propelling or spray formulations) dispensed with a spray can, a nebulizer, or an atomizer can be used. Such formulations can be in the form of a finely comminuted powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the therapeutics also can be delivered in the form of an aerosol spray from a pressured container or dispenser which contains a suitable propellant, e.g., a gas such as carbon dioxide, or a nebulizer. Nasal drops also can be used.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents, bile salts, and filsidic acid derivatives. Transmucosal administration can be accomplished through the use of nasal sprays or suppositories. For transdermal administration, the therapeutics typically are formulated into ointments, salves, gels, or creams as generally known in the art.

In one embodiment, the therapeutics are prepared with carriers that will protect against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Microsomes and microparticles also can be used.

The compounds of the invention may also suitably be administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include liposomally entrapped compositions of the present invention (Epstein, et al., Proc. Natl. Acad. Sci. USA 82:3688-3692 (1985); Hwang et al., Proc. Natl. Acad. Sci. USA 77:4030-4034 (1980).

The compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, the therapeutics identified according to the invention can be formulated for administration to humans or other mammals, for example, in therapeutically effective amounts, e.g., amounts which provide appropriate concentrations of the bioactive agent to target tissue/cells for a time sufficient to induce the desired effect. Additionally, the therapeutics of the present invention can be administered alone or in combination with other molecules known to have a beneficial effect on the particular disease or indication of interest. By way of example only, useful cofactors include symptom-alleviating cofactors, including antiseptics, antibiotics, antiviral and antifungal agents and analgesics and anesthetics.

The effective concentration of the therapeutics identified according to the invention that is to be delivered in a therapeutic composition will vary depending upon a number of factors, including the final desired dosage of the drug to be administered and the route of administration. The preferred dosage to be administered also is likely to depend on such variables as the type and degree of the response to be achieved; the specific composition of another agent, if any, employed; the age, body weight, general health, sex and diet of the patient; the time of administration, route of administration, and rate of excretion of the composition; the duration of the treatment; bioactive agent (such as a chemotherapeutic agent) used in combination or coincidental with the specific composition; and like factors well known in the medical arts. In some embodiments, the therapeutics of this invention can be provided to an individual using typical dose units deduced from the earlier-described mammalian studies using non-human primates and rodents. As described above, a dosage unit refers to a unitary, i.e. a single dose which is capable of being administered to a patient, and which can be readily handled and packed, remaining as a physically and biologically stable unit dose comprising either the therapeutic as such or a mixture of it with solid or liquid pharmaceutical diluents or carriers.

Therapeutics of the invention also include “prodrug” derivatives. The term prodrug refers to a pharmacologically inactive (or partially inactive) derivative of a parent molecule that requires biotransformation, either spontaneous or enzymatic, within the organism to release or activate the active component. Prodrugs are variations or derivatives of the therapeutics of the invention which have groups cleavable under metabolic conditions. Prodrugs become the therapeutics of the invention which are pharmaceutically active in vivo, when they undergo solvolysis under physiological conditions or undergo enzymatic degradation. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992).

The invention also provides a pharmaceutical pack or kit comprising one or more containers filled with one or more of the ingredients of the pharmaceutical compositions of the invention. Associated with such containers can be a notice in the form prescribed by a governmental agency regulating the manufacture, use or sale of pharmaceuticals or biological products, which notice reflects approval by the agency of manufacture, use or sale for human administration, and instructions for administration and dosing. In addition, the compositions of the present invention may be employed in conjunction with other therapeutic compositions.

I. A Chemoenzymatic Approach toward the Rapid and Sensitive Detection of O-GlcNAc Posttranslational Modifications

Introduction

Protein glycoslation is one of the most abundant posttranslational modifications and plays a fundamental role in the control of biological systems. For example, carbohydrate modifications are important for host-pathogen interactions, inflammation, development, and malignancy (Varki, 1993; Lasky, 1996; Capila et al., 2002; Rudd et al., 2001). As part of a broader program to understand the role of protein glycosylation in neuronal communication, O-GlcNAc glycosylation, which is the covalent modification of serine and threonine residues by β-N-acetylglucosamine, was investigated (Wells et al., 2001; Zachara et al., 2002). The O-GlcNAc modification is found in all higher eukaryotic organisms from C. elegans to man and has been shown to be ubiquitous, inducible, and highly dynamic, suggesting a regulatory role analogous to phosphorylation. However, the regulatory nature of the modification (i.e., dynamic, low cellular abundance) also represents a central challenge in its detection and study.

A common method to observe O-GlcNAc involves labeling proteins with β-1,4-galactosyltransferase (GalT), an enzyme that catalyzes the transfer of [³H]galactose from UDP-[³H]galactose to terminal GlcNAc groups (Roquemore et al., 1994). Unfortunately, this approach is expensive, involves handling of radioactive material, and requires exposure times of days to months. Antibodies (Snow et al., 1987; Corner et al., 2001) and lectins (Roquemore et al., 1994) offer alternative means of detection, but they can suffer from weak binding affinity and limited specificity. Described herein is a strategy for the rapid and sensitive detection of O-GlcNAc glycoslated proteins.

Materials and Methods

General Methods: Chemicals and reagents were used without further purification unless otherwise noted. If necessary, reactions were performed under argon atmosphere using anhydrous solvents. Thin layer chromatography was performed using E. Merck silica gel 60 F254 precoated plates and visualized using cerium ammonium molybdate stain. Flash column chromatography was carried out with Silica Gel 60 (230-400 mesh). NMR spectra were obtained on a Varian Mercury 300 instrument. High resolution mass spectra were obtained with a Jeol JMS-600H spectrometer. The peptide TAPTS(O-GlcNAc)TIAPG was synthesized at the Beckman Institute Biopolymer Synthesis Center using standard Fmoc chemistry. The Fmoc-protected, peracetylated O-GlcNAc serine amino acid was synthesized as reported by Seitz et al. (Seitz et al., 1997). Baculovirus preparation and protein expression of CREB in Spodoptera frugiperda (Sf9) cells were performed by Dr. P. Snow at the Beckman Institute Protein Expression Facility at the California Institute of Technology (Lamarre-Vincent et al., 2003). HeLa cell nuclear extracts were prepared according to published procedures (Arts et al., 1997). Y289L and wild-type GalT were expressed and purified as described previously in Ramakrishnan et al., 2002. All protein concentrations were measured using the Bradford assay (Bio-Rad Laboratories, Hercules, Calif.).

General Reagents: Unless otherwise noted, reagents were purchased from the commercial suppliers Fisher (Fairlawn, N.J.) and Sigma-Aldrich (St. Louis, Mo.) and were used without further purification. Protease inhibitors were purchased from Sigma-Aldrich or Alexis Biochemicals (San Diego, Calif.). Bovine GalT, ovalbumin, and α-crystallin were obtained from Sigma-Aldrich. Uridine diphospho-D-[6-³H]galactose, Hyperfilm ECL and Amplify reagent were purchased from Amersham Biosciences (Piscataway, N.J.). WGA lectin was purchased from E-Y Laboratories (San Mateo, Calif.). RL-2 antibody was purchased from Affinity Bioreagents (Golden, Colo.). Alkaline phosphatase was purchased from New England Biolabs (Beverly, Mass.), and bovine serum albumin (BSA) was obtained from Fisher. SuperSignal West Pico chemiluminescence reagents and secondary antibodies were from Pierce (Rockford, Ill.), and the CTD110.6 antibody was purchased from Covance Research Products (Berkeley, Calif.). Nitrocellulose was purchased from Schleicher and Schuell (Keene, N.H.), and PVDF was purchased from Millipore (Bedford, Mass.).

2-Acetonyl-2-deoxy-3,4,5-tri-O-acetyl-β-D-galactopyranose (Ross et al., 2001): Ketone 2 (289 mg, 0.744 mmol) was dissolved in acetonitrile (1.5 mL), and Me₂NH in THF (2.0 M solution, 2.80 mL, 5.60 mmol) was added. The reaction mixture was stirred for 24 h at room temperature (rt). The solvents and reagents were evaporated in vacuo. Flash chromatography on silica gel (1:1 hexanes:EtOAc) gave the monodeacetylated product (136 mg, 0.393 mmol, 53%) as a colorless oil. ¹H NMR (300 MHz, CDCl₃): δ 5.49-5.46 (m, 1H, 1-H), 5.34-5.33 (m, 1H, 4-H), 5.10 (dd, J=12.0, 3.0 Hz, 1H, 3-H), 4.39 (t, J=6.6 Hz, 1H, 5-H), 4.18-4.04 (m, 2H, 6-H₂), 2.84-2.72 (m, 1H, 2-H), 2.62-2.54 (m, 2H, 1′-H₂), 2.17, 2.14, 2.06, 2.01 (4×s, 12H, 3×Ac, 3′-H₃). ¹³C NMR (75 MHz, CDCl₃): δ 207.1, 170.4, 170.3, 170.2, 92.8, 68.7, 66.7, 66.1, 62.3, 40.9, 34.7, 30.4, 20.7, 20.7, 20.1. HRMS (FAB) calcd. for C₁₅H₂₃O₉ [M+H]⁺347.1342, found 347.1342.

Dibenzyl (2-acetonyl-deoxy-3,4,5-tri-O-acetyl-α-D-galactopyranosyl) phophate (3) (Ha et al., 1999; Sim et al., 1993): The deprotected ketone (90 mg, 0.26 mmol) and 1H-tetrazole (91 mg, 1.3 mmol) were dissolved in dichloromethane (3 mL). The reaction mixture was cooled to −30° C. and dibenzyl N,N′-diisopropylphosphamidite (170 μ/L, 0.52 mmol) was added. The reaction mixture was warmed to room temperature (rt) over 30 min and stirred at rt. After 1 h, the reaction mixture was again cooled to −30° C. and mCPBA (229 mg, 1.30 mmol) was added. The mixture was then stirred at 0° C. for 1 h and at rt for 1 h. The reaction was subsequently diluted in dichloromethane, washed twice with 10% Na₂SO₃, once with NaHCO₃, and once with H₂O. The organic phase was dried over MgSO₄, filtered and concentrated. Flash chromatography on silica gel (1:1 hexanes:EtOAc) gave 3 (83 mg, 0.14 mmol, 54%) as a colorless oil. ¹H NMR (300 MHz, CDCl₃): δ7.34-7.32 (m, 10H, arom), 5.86 (dd, J=6.3 Hz, 1H, 1-H), 5.29 (m, 1H, 4-H), 5.15-4.98 (m, 4H, bn), 4.92 (dd, J=2.7, 12.0 Hz, 1H, 3-H), 4.25 (t, J=6.5 Hz, 1H, 5-H), 4.07-3.93 (m, 2H, 6-H₂), 2.90-2.80 (m, 1H, 2-H), 2.35 (d, J=7.2 Hz, 2H, 1′-H₃), 2.09, 1.95, 1.91, 1.87 (4×s, 12H, 3×ac, 3′-H₂). ³¹P NMR (121 MHz, CDCl₃): δ-1.31. ¹³C NMR (75 MHz, CDCl₃): δ205.7, 170.0, 170.0, 169.8, 128.6, 128.5, 128.5, 127.9, 97.7 (d), 69.6 (d), 69.5, 68.3, 68.0, 65.9, 61.7, 39.1, 34.4 (d), 29.9, 20.6, 20.6, 20.5. HRMS (FAB): calcd. for C₂₉H₃₆O₁₂P [M+H]⁺607.1945, found 607.1924.

Uridine 5′-diphospho-2-acetonyl-2deoxy-α-D-galactopyranose diammonium salt (1) (Wittmann et al., 1997; Hitchcock et al., 1998): A solution of dibenzyl phosphate 3 (80 mg, 0.13 mmol) and tri-n-octylamine (35 μL) in methanol (10 mL) was hydrogenolyzed in the presence of 10% Pd/C (100 mg) under 1 atm H₂ for 20 h. The mixture was filtered, concentrated, dried and directly used in the next step. UMP-morpholidate 4-morpholine- N,N′-dicyclohexylcarboxamidine salt (36 mg, 0.198 mmol) was added and the mixture was evaporated three times from anhydrous pyridine (1.5 mL). The mixture was dissolved in pyridine (1.0 mL), 1H-tetrazole (28 mg, 0.40 mmol) was added, and the solution was stirred for three days at rt. After evaporation of the solvent, the reaction product was dissolved in a mixture of MeOH/water/TEA (2 mL/0.8 mL/0.4 mL) and stirred for 24 h. The residue was then dissolved in water and dichloromethane, and the organic phase was extracted twice with water. The aqueous phases were combined and lyophilized. The residue was purified on a Bio-Gel P2 (extra fine) column (1.5×80 cm), and eluted with 0.1 M NH₄HCO₃ at a flow rate of 0.6 mL/min. Lyophilization of the desired fractions (determined by HPLC Varian Microsorb C18, 100 mM NH₄HCO₃, 4.1 min) gave 1 (38.7 mg, 0.060 mmol, 45%) as a colorless powder. ¹H NMR (300 MHz, D₂O): δ 7.96 (d, J=8.1 Hz, 1H, 6″-H), 5.97-5.94 (m, 2H, 5″-H, 1′-H), 5.55 (dd, J=7.8, 3.3 Hz, 1H, 1-H), 4.36-4.33 (m, 2H, 2′-H, 3′-H), 4.26-4.24 (m, 1H, 4′-H), 4.21-4.17 (m, 2H, 5′-H₂), 4.13 (t, J=5.1 Hz, 1H, 5-H), 3.88 (m, 1H, 4-H), 3.79-3.69 (m, 3H, 3-H, 6-H₂), 2.79-2.75 (m, J=4.2 Hz, 2H, 1′″-H₂), 2.53 (m, 1H, 2-H), 2.24 (s, 3H, 3′″-H₃). ³¹P NMR (121 MHz, CDCl₃): δ-10.74 (d, J=19.5 Hz), −12.06 (d, J=20.1 Hz). ¹³C NMR (75 MHz, D₂O): δ214.3, 166.3, 151.9, 141.8, 102.9, 96.5, 88.6, 83.6, 74.0, 72.1, 69.9, 68.2, 65.1, 63.9, 61.6, 43.5, 41.6, 30.3. HRMS(EI) calcd. for C₁₈H₂₇O₁₇N₂P₂ [M−H]⁻605.0785, found 605.0803.

Labeling of the O-GlcNAc Peptide: The peptide TAPTS(O-GlcNAc)TIAPG (10 μM was dissolved in 25 mM MOPS buffer, pH 6.7 containing 5 mM MnCl₂ and 8 μM reference peptide (ThermoFinnigan, San Jose, Calif.). Ketone analogue 1 and mutant Y289L GalT were added to final concentrations of 1 mM and 100 ng/μL, respectively. Prior to enzyme addition, an aliquot of the reaction was removed as an initial time point for LC-MS analysis. Reactions were incubated at 4° C. for 6 h, after which an aliquot of the reaction mixture was removed for product analysis by LC-MS. The remainder of the reaction was diluted 5-fold into PBS (final concentration: 10.1 mM Na₂HPO₄, 1.76 mM KH₂ HPO₄, 137 mM NaCl, 2.7 mM KCl, pH 6.7), and N-(aminooxyacetyl)-N′-(D-biotinoyl) hydrazine (Molecular Probes, Eugene, Oreg.) was added to a final concentration of 12 mM. After 8 h at 25° C., the extent of biotin-oxime product was measured by LC-MS. A 6000:1 molar ratio of aminooxy biotin was optimal for complete conversion to the oxime product. Labeling reactions with wild-type GalT were performed identically, with the exception that reactions were incubated at 37° C. for 12 h.

LC-MS monitoring of O-GlcNAc peptide labeling reactions: Liquid chromatography and mass spectrometry (LC-MS) were performed on an LCQ Classic ion trap mass spectrometer (ThermoFinnigan, San Jose, Calif.) interfaced with a Surveyor HPLC system (ThermoFinnigan, San Jose, Calif.). Approximately 10 pmoles of peptide from each labeling reaction was loaded onto a Luna column (2 mm i.d.×50 mm) prepacked with 3 μm 100 Å C18 RP particles. Flow rate was maintained at 190 μL/min with a gradient optimized for separation of the O-GlcNAc peptide from labeled products. LC buffer A consisted of 2% CH₃CN in 0.1M aqueous AcOH and buffer B consisted of 90% CH₃CN in 0.1M aqueous AcOH. The gradient consisted of 0-3 min, 2% B; 3-6 min, 2-11% B; 11-14.5 min 11-27.5% B, 14.5-18 min 27.5-100% B; 18-22 min 100% B where the initial 5 minutes of flow were diverted to waste in order to avoid contamination of the mass spectrometer with salts. The LCQ was operated in automated mode using Xcalibur™ software. The electrospray voltage was 4.5 kV and the heated capillary was 200° C. Ion injection time was set at 200 ms for full MS scan mode of operation (3 microscans per scan). The ion selection window was set at 500-1700 m/z for all experiments.

As monitored by LC-MS, complete conversion of the peptide to the desired ketone-labeled product was observed. For the aminooxy biotin reaction, formation of the oxime product was monitored using an extracted ion chromatogram within the mass range 1319.0-1321.0 m/z and 1633.0-1635.5 m/z, which was generated post-acquisition via the Xcalibur™ software. No appreciable amounts of the unbiotinylated starting material were observed after 8 h. Mass spectrometric analysis confirmed the identity of each product.

The extent of conversion to ketone-labeled peptide was analyzed by measuring peak areas for the starting material (peak a) and product (peak b) using Xcalibur™ software, under the assumption that the O-GlcNAc peptide and its ketone-labeled analogue had similar ionization potentials. Approximately 1.5% of the desired product was formed with the wild-type GalT.

Labeling of CREB protein: Recombinant O-GlcNAc glycosylated CREB was generated by coexpression of CREB with O-GlcNAc glycosyltransferase in Sf9 cells as described previously (Lamarre-Vincent et al., 2003). 500 ng of CREB in 20 mM HEPES pH 7.9, 100 mM KCl, 0.2 mM EDTA, 15% glycerol was added to 50 mM MOPS pH 6.45 containing 5 mM MnCl₂ and 0.25 mU/μL alkaline phosphatase (Unverzagt et al., 1990). Analogue 1 and Y289L GalT were then added to final concentrations of 1 mM and 40 ng/μL, respectively. Control reactions without enzyme or analogue 1 were treated identically. Following incubation at 12 h at 4° C., the reactions were diluted 5-fold into PBS containing protease inhibitors (5 μg/mL pepstatin, 5 μg/mL chymostatin, 20/μg/mL leupeptin, 20 μg/mL aprotinin, 20 μg/mL antipain, 0.2 mM PMSF). Aminooxy biotin was added to a final concentration of 2 mM, and the biotinylation reactions were incubated with gentle shaking for 12 h at 37° C. Reactions were aliquoted for analysis and stopped by boiling in SDS-PAGE loading dye. Proteins were resolved by 10% SDS-PAGE, electrophoretically transferred to nitrocellulose, and probed with streptavidin-HRP.

Nitrocellulose blots were blocked for 1 h at rt using 3% periodated-BSA (Glass et al., 1981) in PBS, rinsed once with TBS (50 mM Tris.HCI, 150 mM NaCl, pH 7.4) containing 0.05% (v/v) tween-20, and probed with streptavidin-HRP (1:2500 to 1:5000) in TBS-0.05% tween for 1 h at rt. In some cases, blots were probed for 1 h with streptavidin-HRP, rinsed several times with TBS-0.05% tween, and reprobed with another aliquot of streptavidin-HRP. After probing with streptavidin, membranes were rinsed and washed 5×10 min with TBS-0.1% tween containing 0.05% BSA. Streptavidin-HRP signal was visualized by chemiluminescence upon exposure to film. After streptavidin visualization, membranes were stripped in 5 mM Na₂HPO₄ pH 7.5, 2% SDS, and 2 mM βME, for 45 min at 60° C., rinsed several times with dH₂O, and re-probed with α-CREB antibody as previously described (Lemarre-Vincent et al., 2003) with the modification that the antibody was used at a concentration of 1:400.

Labeling reactions with CREB expressed in E. coli were performed identically. To generate the bacterial protein, rat CREB cDNA was cloned into the prokaryotic expression vector pET23b(+) (Novagen, Madison, Wis.) using HindIII and NdeI restriction endonucleases. Electrocompetent BL21(DE3) cells were electroporated and grown in Luria-Bertani media supplemented with 100 mg/L ampicillin. Protein expression was induced with 0.3 mM isopropyl-β-D-thiogalactopyranoside. Recombinant CREB was purified using Ni-NTA agarose (Qiagen, Valencia, Calif.) as described previously (Lemarre-Vicent et al., 2003).

Strong, selective labeling of glycosylated CREB was observed upon treatment with both Y289L GalT and analogue 1. With larger quantities of protein, a faint background signal was observed, which was presumably due to the non-specific interaction of aminooxy biotin with the protein. Importantly, the background signal was readily diagnosed using control reactions in the absence of enzyme or analogue 1. In the case of E. coli CREB, for example, a weak background signal was observed over time, but no selective enhancement of signal was seen in the presence of both enzyme and analogue 1, indicating that bacterially expressed CREB was not GlcNAc glycosylated.

Labeling of α-crystallin: Bovine lens α-crystallin (a mixture of A and B chains) was resolved by SDS-PAGE electrophoresis and Coomassie-stained with standards in order to quantify the amount of A chain in the mixture. For reactions, 8.7 μg of α-crystallin (6.5 μg of A chain) in 20 mM HEPES pH 7.9 was added to 50 mM MOPS pH 6.45 containing 5 nM MnCl₂ and 0.25 mU/μL alkaline phosphatase. Analogue 1 and Y289L GalT were added to final concentrations of 1 mM and 10 ng/μL, respectively. Reactions were incubated at 4° C. for 18 h and then diluted 5-fold with PBS pH 6.7, protease inhibitors, and aminooxy biotin (6.5 mM final concentration). Biotinylation reactions were incubated with gentle shaking at 25° C. for 12 h. The molar ratio of biotin to α-crystallin was adjusted to minimize background signal, while maintaining reactivity over a reasonable time period (e.g., a 4000:1 molar ratio). After biotinylation, reactions were aliquoted for analysis and subsequently boiled in SDS-PAGE loading dye. Proteins were resolved by 15% SDS-PAGE transferred to nitrocellulose, and probed with streptavidin-HRP or stained with Coomassie Brilliant Blue. Blotting with streptavidin-HRP was performed as described above and produced a strong signal within 30 min. In contrast, tritium labeling required 8 days to obtain a moderate signal.

UDP-[³H]galactose labeling of α-crystallin: ³H-labeling was performed essentially as described (Roquemore et al., 1992; Roquemore et al., 1994). Briefly, 8.7 μg of α-crystallin (6.5 μg of A chain) in 20 mM HEPES pH 7.9 was added to 10 mM HEPES pH 7.9 containing 5 mM MnC₂ and protease inhibitors. UDP-[³H]-galactose was added to a final concentration of 0.03 μCi/μL, and the reaction was initiated with the addition of 25 mU autogalactosylated bovine β1,4-galactosyltransferase (Roquemore et al., 1994). Reactions were incubated at 37° C. for 1 h 15 min. Reactions were subsequently aliquoted for analysis and stopped by boiling with SDS-PAGE loading dye. Proteins were resolved by 15% SDS-PAGE, stained with Coomassie Brilliant Blue, incubated with Amplify reagent, and dried for subsequent exposure to Hyperfiln MP at −80° C.

Western blotting of α-crystallin using antibodies RL-2 and CTD110.6: α-Crystallin, and appropriate positive and negative controls were resolved by 15% SDS-PAGE. All Western blotting steps were performed at rt unless otherwise noted. Western blotting with the RL-2 antibody was performed according to reported methods (Konrad et al., 2000) with minor changes suggested by the manufacturer to reduce background noise. α-Crystallin and controls were electrophoretically transferred to nitrocellulose blots, and the blots were blocked for 1 h in 5% BSA in high salt (250 mM) TBS-1% tween-20 (hsTBS-T). RL-2 antibody, at a concentration of 1:2000, was subsequently added in blocking buffer and blots were incubated for 1.5-2 h. Blots were then rinsed with hsTBST and washed 6×5 min. Secondary goat anti-mouse IgG antibody was applied at a concentration of 1:10,000 in hsTBS-T containing 1% BSA. After 1 h, blots were rinsed and washed as described before, followed by chemiluminescence detection on film. Western blotting with the CTD 110.6 antibody was performed according to manufacturer's recommendations. Briefly, α-crystallin and controls were transferred to PVDF and washed 2×15 min with TBS-0.1% tween-20 (TBST). Blots were blocked in TBST containing 3% BSA for 1 h, rinsed 2× with TBST, and probed with CTD110.6 (1:2500) in blocking buffer for 1 h. Blots were then rinsed 2× with TBST and washed 2×5 min. Secondary goat anti-mouse IgM antibody was applied at a concentration of 1:10,000 in blocking buffer for 1 h, and blots were subsequently rinsed with TBST and washed 5×5 min before chemiluminescence detection on film.

WGA lectin blotting of α-crystallin: WGA western blotting was performed essentially as described (Roquemore et al., 1994; Freeze et al., 1999). Briefly, α-crystallin and controls were resolved by 15% SDS-PAGE and electrophoretically transferred to nitrocellulose. Blots were blocked for 1 h in 3% periodatetreated BSA in PBS, rinsed 2×15 min with PBS-0.05% tween-20 (PBST), and probed for 2 h with WGA-HRP (1:8000 in PBST). Subsequently, blots were rinsed with PBST, washed 3×10 min, then 3×20 min before chemiluminescence detection on film.

Results

A strategy for the rapid and sensitive detection of O-GlcNAc glycoslated proteins is described herein. The approach capitalizes on the substrate tolerance of GalT, which allows for chemoselective installation of an unnatural ketone functionality to O-GlcNAc modified proteins. The ketone moiety has been well-characterized in cellular systems as a neutral, yet versatile, chemical handle (Cornish et al., 1996; Mahal et al., 1997; Datta et al., 2002). Here, it serves as a unique marker to “tag” O-GlcNAc glycosylated proteins with biotin. Once biotinylated, the glycoconjugates can be readily detected by chemiluminescence using streptavidin conjugated to horseradish peroxidase (HRP).

UDP analogue 1 was designed on the basis of previous biochemical and structural studies of GalT. The ketone functionality was appended at the C-2 position of the galactose ring because GalT has been shown to tolerate unnatural substrates containing minor substiutions at the C-2 positions, including 2-deoxy, 2-amino, and 2-N-acetyl substituents (Qian et al., 2001; Wong et al., 1995). Moreover, 2-deoxy-Gal was transferred at rates comparable to Gal, whereas 3-, 4, and 6-deoxy-Gal were transferred at reduced rates. Analysis of the crystal structures of GalT complexed with UDP-GalNAc revealed that the C-2 N-acetyl moiety is accommodated in a shallow pocket within the active site Ramakrishnan et al., 2002). Importantly, the single Y289L mutation enlarges the binding pocket and enhances the catalytic activity toward GalNAc substrates without compromising specificity (Ramakrishnan et al., 2002).

Analogue 1 was synthesized from the previously reported ketone 2 (Hang et al., 2001) as shown in Scheme 1 (Conditions: (a) Me₂NH, THF (53%); (b) (BnO)₂PNiPr₂, then mCPBA (54%); (c) Pd/C, H₂, tri-n-octylamine; (d) UMP-morpholidate, 1H-tetrazole, pyr; (e) TEA, H₂O/MeOH (45%, three steps)). Selective anomeric deacetylation followed by treatment with (BnO)₂PNiPr₂ (Sim et al., 1993) afforded the phosphite, which was directly oxidized with mCPBA (Ha et al., 1999) to produce dibenzyl phosphate 3. Hydrogenolytic debenzylation yielded the unprotected phosphate as the trioctylammonium salt, which was coupled with UMP-morpholidate in pyridine (Wittmann et al., 1997) to provide molecule 1 upon deacetylation with TEA.

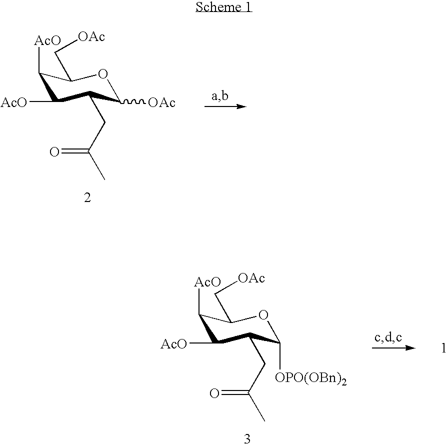

With analogue 1 in hand, the ability of GAlT to label the peptide TAPTS(O-GlcNAc)TIAPG, which encompasses an O-GlcNAc modification site within the protein CREB (Lamarre-Vincent et al., 2003), was examined. Using wild-type GalT, only partial transfer of the keto-sugar was observed by LC-MS (˜1.5%). However, the Y289L mutant showed greater activity and afforded complete conversion after 6 h at 4° C. Subsequent reaction of the ketone-labeled peptide with the aminooxy biotin derivative, N-(aminooxyacetyl)-N′-(D)-biotinoyl) hydrazine, under mild conditions (pH 6.7 buffer, 8 h, 25° C.) gave complete formation of the corresponding O-alkyl oxime.

Having demonstrated the labeling of a peptide, the strategy was applied to the O-GlcNAc glycosylated protein CREB. Recombinant CREB from Sf9 cells (Lamarre-Vincent et al., 2003) was incubated with 1 and Y289L GalT for 12 h at 4° C. Following reaction with aminooxy biotin, the mixture was resolved by SDS-PAGE, transferred to nitrocellulose, and probed with streptavidin-HRP.

Strong labeling of CREB was observed by chemiluminescence within seconds of exposure to film. In contrast, no signal was observed over the same time period for unglycosylated CREB (from E. coli) or when reactions were performed in the absence of either 1 or enzyme, demonstrating the selectivity of the transfer.

The sensitivity of the approach using the challenging target, α-crystallin, was determined. Detection of the O-GlcNAc moiety on α-crystallin has been reported to be particularly difficult due to its low stoichiometry of glycosylation (˜10%) and the presence of only one major modification site (Chalkley et al., 2001; Haynes et al., 2000). It was determined that the existing methods, such as wheat-germ agglutinin (WGA) lectin (Roquemore et al., 1994) and the O-GlcNAc-specific antibodies RL-2 (Snow et al., 1987) and CTD110.6 (Comer et al., 2001), failed to detect any O-GlcNAc modification on α-crystallin, even when 10 μg of α-crystallin was used. In contrast, the approach described herein enabled detection of the O-GlcNAc modification within minutes using 0.75 μg of α-crystallin. For comparison, tritium labeling with wild-type GalT required 8 days of exposure to film for a weaker signal. The approach described herein represents at least a 380-fold enhancement in signal over traditional methods.

Recently, Vocadlo et al. have reported the extension of their Staudinger methodology to O-GlcNAc glycosylated proteins (Vocadlo et al., 2003). The strategy described here complements the metabolic labeling approach and is distinct in several key respects. First, the use of an engineered GalT, and 1 enables near stoichiometric labeling, resulting in higher sensitivity. Enhanced sensitivity is crucial in studying O-GlcNAc as the regulatory nature of the modification means that it is often present only in low cellular abundance. Second, the use of an engineered GalT rather than the native O-GlcNAc glycosyltransferase allows one to capture the glycosylated species directly and avoid perturbation of metabolic pathways. Thus, the approach should permit the observation of O-GlcNAc signaling pathways after cellular stimulation, an important frontier in the field.

### BIBLIOGRAPHY

Andree, P. J. and Berliner L. J., Biochim. Biophys. Acta, 544:489-495 (1982).
Arts et al. Nucleic Acids Res., 25, 311-317 (1997).
Berliner, L. J. and Robinson, R. D., Biochemistry 21:6340-6343 (1982).
Berliner, L. J. et al., Mol. Cell. Biochem., 62:37-42 (1984).
Bundgard, Design of Prodrugs, Elsevier, Amsterdam (1985).
Capila, I.; Lindhardt, R. J., Angew. Chem., Int. Ed., 41, 391412 (2002).
Chalkley, R. J.; Burlingame, A. L., J. Am. Soc. Mass Spectrum 12, 1106-1113 (2001).
Comer et al., Anal. Biochem. 293, 169-177 (2001).
Cornish et al., J. Am. Chem. Soc. 118, 8150-8151 (1996).
Datta et al., J. Am. Chem. Soc., 124, 5652-5653 (2002).
Do, K. Y. et al., J. Biol. Chem., 270:18477-18451 (1995).
Epstein, et al., Proc. Natl. Acad. Sci. USA, 82:3688-3692 (1985).
Freeze, H. H. In Current Protocols in Molecular Biology; Ausubel, F. M., Brent, R., Kingston, R. E., Moore, D. D., Seidman, 1. G., Smith, J. A., Struhl, K., Eds.; John Wiley & Sons, Inc.: New York, 1999; Vol. 3 pp 17.7.1-17.7.8.
Glass et al., Anal. Biochem., 115, 219-224 (1981).
Glockshuber, R. et al., Biochemistry 29:1362 (1990).
Green & Wuts, Protective Groups in Organic Synthesis (John Wiley & Sons) (1991).
Ha et al., J. Am. Chem. Soc., 121, 8415-8426 (1999).
Halin et al., Nat. Biotechnol., 20:264 (2002).
Hammerling et al., In: “Monoclonal Antibodies and T-Cell Hybridomas,” Elsevier, N.Y., pp. 563-681 (1981).
Hang, H. C.; Bertozzi, C. R., J. Am. Chem. Soc. 123, 1242-1243 (2001).
Hanover, FASEB, 15:1865 (2002).
Haynes, P. A.; Aebersold, R. Anal Chem., 72, 5402-5410 (2000).
Hill, UCLA Forum Med. Sci., 21:63-86 (1979).
Hitchcock et al., J. Am. Chem. Soc., 120, 1916-1917 (1998).
Hwang et al., Proc. Natl. Acad. Sci. USA 77:4030-4034 (1980).
Khidekel et al., J. Am. Chem. Soc. 125:16162-16163 (2003).
Kennedy et al., Clin. Chim. Acta. 70:1-31 (1976).
Kohler et al., Eur. J. Immunol., 6:511 (1976).
Kohler et al, Eur. J. Immunol. 6:292 (1976).
Kohler, G. and Milstein, C., Nature, 256:495 (1975).
Konrad et al., Biochem. Biophys. Res. Commun., 267, 26-32 (2000).
Kumamoto et al., Nat. Biotechnol. 20:64 (2002).
Lamarre-Vincent, N.; Hsieh-Wilson, L. C., J. Am. Chem. Soc. 125, 6612-6613 (2003).
Lasky, L. A., Annu. Rev. Biochem., 64113-139 (1995).
Mahal et al., Science 276, 1125-1128 (1997).
Morrison, S. L. et al., Proc. Nat'l Acad. Sci., 81:6851 (1984).
Palcic, M. M and Hindsgaul, O., Glycobiology. 1:205-209 (1991).
Qian et al., Glycochemistry: Principles. Synthesis, and Application, 535-565 (2001).
Ramakrishnan, B. et al., J. Biol. Chem. 276:37665-37671 (2001).
Ramakrishnan, B.; Qasba, P. K., J. Biol. Chem. 277, 20833-20839 (2002).
Ramasamy et al., J. Mol. Biol., 331(5):1065 (2003).
Remington: The Science and Practice of Pharmacy, 19^{th} Ed., Easton, Pa.: Mack Publishing Co. (1995).
Remington's Pharmaceutical Sciences, (Gennaro, A., ed.), Mack Pub., 1990.
Roquemore et al., J. Biol. Chem., 267, 555-563 (1992).
Roquemore et al., Methods Enzmol., 230, 443-460 (1994).
Ross et al., J. Chem. Soc.-Perkin Trans., 1, 72-81 (2001).
Rudd et al., Science, 291, 2370-2376 (2001).
Schurs et al., Clin. Chim. Acta, 81:1-40 (1977).
Seitz et al., J. Am. Chem. Soc. 119, 8766-8776 (1997).
Silverman, The Organic Chemistry of Drug Design and Drug Action, Academic Press, San Diego, Calif., pp. 352-401 (1992).
Sim et al., J. Am. Chem. Soc., 115, 2260-2267 (1993).
Snow et al., J. Cell Biol. 104, 1143-1156 (1987).
Svensson et al., Protein Science, 12:2794-2804 (2003).
Torres and Hart, J. Biol. Chem., 259:3308 (1984).
Unverzagt et al., J. Am. Chem. Soc., 112, 9308-9309 (1990).
Varki, A., Glycobiology, 3, 97-130 (1993).
Vocadlo et al., Proc. Natl. Acad. Sci. U.S.A., 100, 9116-9121 (2003).
Wands et al., Gastroenterology, 80:225-232 (1981).
Ward, E. S. et al., Nature, 341:544 (1989).
Wells et al., Science, 291, 2376-2378 (2001).
Winter, G. and Milstein C., Nature, 349:295 (1991).
Wittman, V.; Wong, C.-H., J. Org. Chem. 62, 2144-2147 (1997).
Wong et al., Angew. Chem., Int. Ed. Engl., 34, 521-546 (1995).
Zachara et al., Chem. Rev., 102, 431-438 (2002).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A targeted glycoconjugate comprising a bioactive agent and a targeting compound, wherein the bioactive agent and targeting compound are joined by a modified saccharide compound.

2. The glycoconjugate of claim 1 wherein the bioactive agent comprises a polypeptide; releasing factor; releasing factor inhibitor; carbohydrate; nucleic acid; vaccine; anti-antibiotic; antiviral agent; anti-fungal agent; analgesics anesthetic; anti-helminthic; anti-arthritic agent; anti-asthmatic agent; anticonvulsant; antidepressant; anti-diabetic agent; anti-diarrheal; anticonvulsant; antihistamine; anti-inflammatory agent; toxin, anti-migraine preparation; anti-nauseant; anticancer agent; anti-parkinsonism drug; anti-psychotic; antipyretic; anti-spasmodic; anti-cholinergic; sympathomimetic; xanthine derivative; cardiovascular agent; anti-arrhythmic; anti-hyperlipidemic agent; anti-hypertensive; diuretic; anti-diuretic; receptor agonist; receptor antagonist; vasodilator; central nervous system stimulant; vasoconstrictor; cough and cold preparation; enzyme inhibitor; hormone; hypnotic; agent; muscle relaxant; parasympatholytic; central nervous system stimulant; diuretic; hypnoticsleukotriene inhibitor; mitotic inhibitor; muscle relaxant; genetic material; psychostimulant; sedative; anabolic agent; vitamin; herbal remedy; anti-metabolic agent; anxiolytic; attention deficit disorder (ADD) drug; attention deficit hyperactivity disorder (ADHD) drug; neuroleptic agent; or tranquilizer.

3. The glycoconjugate of claim 1, wherein the targeting compound comprises a glycoprotein, glycolipid or carbohydrate.

4. The glycoconjugate of claim 1, wherein the targeting compound comprises GlcNAc.

5. The glycoconjugate of claim 1, wherein the targeting compound is a receptor ligand or an antibody.

6. The glycoconjugate of claim 5, wherein the antibody is a polyclonal antibody.

7. The glycoconjugate of claim 5, wherein the antibody is a monoclonal antibody.

8. The glycoconjugate of claim 1, wherein the modified saccharide compound comprises galactose, glucose (Glc), D-deoxy-Glc, arabinose, GalNAc or GlcNAc.

9. The glycoconjugate of claim 8, wherein the modified saccharide compound further comprises a reactive functional group.

10. The glycoconjugate of claim 9, wherein the reactive functional group comprises an amino, hydroxy, carboxyl, thiol, phosphate, phosphinate, ketone, sulfate or sulfinate group.

11. The glycoconjugate of claim 9, wherein the reactive functional group is attached to the C2 position of the saccharide ring.

12. The glycoconjugate of claim 1 wherein the modified saccharide is galactose with a ketone moiety attached at the C2 position of the galactose ring.

13. A method for the treatment or detection of a disease or disorder comprising, administering to a subject in need thereof an effective amount of the glycoconjugate of claim 1.

14. A method of delivering one or more bioactive agents comprising administering to a subject the glycoconjugate of claim 1.

15. A method of vaccinating a subject against a disease comprising administering to the subject an immunologically effective amount of the glycoconjugate of claim 1.

16. The method of claim 13, wherein the bioactive agent comprises a polypeptide; releasing factor; releasing factor inhibitor; carbohydrate; nucleic acid; vaccine; anti-antibiotic; antiviral analgesics anesthetic; anti-helminthic; anti-arthritic agent; anti-asthmatic agent; anticonvulsant; antidepressant; anti-diabetic agent; anti-diarrheal; anticonvulsant; antihistamine; anti-inflammatory agent; toxin, anti-migraine preparation; anti-nauseant; anticancer agent; anti-parkinsonism drug; anti-pruritic; anti-psychotic; antipyretic; anti-spasmodic; anti-cholinergic; sympathomimetic; xanthine derivative; cardiovascular agent; anti-hyperlipidemic agent; anti-hypertensive; diuretic; anti-diuretic; receptor agonist; receptor antagonist; vasodilator; central nervous system stimulant; vasoconstrictor; cough and cold preparation; enzyme inhibitor; hormone; hypnotic; immunosuppressive agent; muscle relaxant; parasympatholytic; central nervous system stimulant; diuretic; hypnoticsleukotriene inhibitor; mitotic inhibitor; muscle relaxant; genetic material; psychostimulant; sedative; anabolic agent; vitamin; herbal remedy; anti-metabolic agent; anxiolytic; attention deficit disorder (ADD) drug; attention deficit hyperactivity disorder (ADHD) drug; neuroleptic agent; or tranquilizers.

17. The method of claim 13, wherein the targeting compound comprises a glycoprotein, glycolipid or carbohydrate.

18. The method of claim 13, wherein the targeting compound comprises GlcNAc.

19. The method of claim 13, wherein the targeting compound is a receptor ligand or an antibody.

20. The method of claim 19, wherein the antibody is a polyclonal antibody.

21. The method of claim 19, wherein the antibody is a monoclonal antibody.

22. The method of claim 13, wherein the modified saccharide compound comprises galactose, glucose (Glc), D-deoxy-Glc, arabinose, GalNAc or GlcNAc.

23. The method of claim 13, wherein the modified saccharide compound comprises a reactive functional group.

24. The method of claim 23, wherein the functional group comprises an amino, hydroxy, carboxyl, thiol, phosphate, phosphinate, ketone, sulfate or sulfinate group.

25. The method of claim 23, wherein the functional group is attached to the C2 position of the saccharide ring.

26. The method of claim 13, wherein the modified saccharide is galactose with a ketone moiety attached at the C2 position of the galactose ring.

27. The method of claim 13, wherein the disease or disorder comprises cancer; inflammatory disease or disorder; a hyperproliferative disorder; hormone deficiency disease; hormone abnormality due to hypersecretion; infectious disease; bacterial infection; viral infection; fungal infection; parasitic infection; cardiovascular disease or disorders; genetic disease; autoimmune disease; allergic reaction or conditions; organ rejection or graft-versus-host disease; immune deficiency disease.

28. The method of claim 13, wherein the subject is a mammal.

29. The method of claim 28, wherein the mammal is a human.

30. A method to synthesize the glycoconjugate of claim 1 comprising: (a) incubating a reaction mixture comprising a 4)-galactosyltransferase I or a mutant thereof with a targeting compound and a donor molecule comprising a modified saccharide residue so as to form a targeting-modified saccharide compound; and (b) incubating the targeting-modified saccharide compound formed in (a) and a bioactive agent under conditions effective to generate a covalent bond between the modified saccharide and the bioactive agent.

31. A method to synthesize the glycoconjugate of claim 1 comprising: (a) incubating a reaction mixture of a donor molecule comprising a modified saccharide residue and a bioactive active agent under conditions effective to generate a covalent bond between the modified saccharide and the bioactive agent; and (b) incubating a reaction mixture comprising a 4)-galactosyltransferase I or a mutant thereof with the modified saccharide-bioactive agent compound formed in (a) with a targeting compound so as to form the glyconjugate.

32. The method of claim 30 wherein the modified saccharide compound comprises galactose, glucose (Glc), or arabinose.

33. The method of claim 30 wherein the modified saccharide compound comprises a reactive functional group.

34. The method of claim 33, wherein the functional group comprises an amino, hydroxy, carboxyl, thiol, phosphate, phosphinate, ketone, sulfate or sulfinate group.

35. The method of claim 33, wherein the functional group is attached to the C2 position of the saccharide ring.

36. The method of claim 30 wherein the modified saccharide is a galactose residue with a ketone moiety attached at the C2 position of the galactose ring.

37. The method of claim 30 wherein the targeting compound comprises a glycoprotein, glycolipid or carbohydrate.

38. The method of claim 30 wherein the targeting compound comprises GlcNAc.

39. The method of claim 30 wherein the targeting compound is a receptor ligand or an antibody.

40. The method of claim 39, wherein the antibody is a polyclonal antibody.

41. The method of claim 39, wherein the antibody is a monoclonal antibody.

42. The method of claim 30, wherein the bioactive agent comprises a polypeptide; releasing factor; releasing factor inhibitor; carbohydrate; nucleic acid; vaccine; anti-antibiotic; antiviral agent; agent; analgesics anesthetic; anti-helminthic; anti-arthritic agent; anti-asthmatic agent; anticonvulsant; antidepressant; anti-diabetic agent; anti-diarrheal; anticonvulsant; antihistamine; anti-inflammatory agent; toxin, anti-migraine preparation; anti-nauseant; anticancer agent; anti-parkinsonism drug; anti-pruritic; anti-psychotic; antipyretic; anti-spasmodic; anti-; sympathomimetic; xanthine derivative; cardiovascular agent; anti-arrhythmic; agent; anti-hypertensive; diuretic; anti-diuretic; receptor agonist; receptor antagonist; vasodilator; central nervous system stimulant; vasoconstrictor; cough and cold preparation; enzyme inhibitor; hormone; hypnotic; hormonolytic; immunosuppressive agent; muscle relaxant; parasympatholytic; central nervous system stimulant; diuretic; hypnoticsleukotriene inhibitor; mitotic inhibitor; muscle relaxant; genetic material; psychostimulant; sedative; anabolic agent; vitamin; herbal remedy; anti-metabolic agent; anxiolytic; attention deficit disorder (ADD) drug; attention deficit hyperactivity disorder (ADHD) drug; neuroleptic agent; or tranquilizers.

43. A pharmaceutical composition comprising the glycoconjugate of claim 1 and a pharmaceutically acceptable carrier.

44. A kit comprising the glycoconjugate of claim and instructions for use in a therapeutic or diagnostic method.

45. A glycoconjugate according to claim 1 for use in medical therapy.

46-48. (canceled)
